(19) United States
(12) Patent Application Publication
(10) Pub. No.: US 2024/0150831 A1
(43) Pub. Date: May 9, 2024

(54) NUCLEIC ACID-BASED SELF-ASSEMBLED COMPLEX FOR RAMAN DETECTION OF TARGET NUCLEIC ACID AND USE THEREOF

(71) Applicant: MODERN VALUE INC., Gyeonggi-do (KR)

(72) Inventors: Dong-woo LEE, Gyeonggi-do (KR); Su-min PARK, Gyeonggi-do (KR); Sung Su KIM, Gyeonggi-do (KR); Yujae HYUN, Gyeonggi-do (KR)

(21) Appl. No.: 18/278,457

(22) PCT Filed: Feb. 23, 2022
(86) PCT No.: PCT/KR2022/002652

§ 371 Date: Aug. 23, 2023

(30) Foreign Application Priority Data
Feb. 23, 2021 (KR) 10-2021-0024393

(51) Int. Cl. C12Q 1/6876; C12Q 1/6825

(52) U.S. Cl. CPC C12Q 1/6876; C12Q 1/6825

(57) ABSTRACT

The present invention relates to a nucleic acid based self-assembled complex for Raman detection of a target nucleic acid, and use thereof. The present invention provides a nucleic acid based self-assembled complex of turn-off based way for detecting a target nucleic acid, which has a structural stability so as produce a reproducible scattering light signal regardless of Brownian motion in liquid and relates to a method of detecting a target nucleic acid as an optical signal such as a Raman signal through a nucleic acid based self-assembled complex performing Brownian motion in a liquid phase.

### TECHNICAL FIELD

The present invention relates to a nucleic acid based self-assembled complex of turn-off based way for detecting a target nucleic acid, which has a structural stability so as produce a reproducible scattering light signal regardless of Brownian motion in liquid and relates to a method of detecting a target nucleic acid as an optical signal such as a Raman signal through a nucleic acid based self-assembled complex being in Brownian motion in a liquid phase.

### BACKGROUND ART

Self-assembly refers to formation of a structure by spontaneous non-covalent bonding between components. Molecular-level self-assembly refers to designing building blocks that form a stable and regular structure by spontaneous bonding. Self-assembly can be found in biological systems, and actin filaments, viruses, and chromatin, which use molecules such as nucleotides, proteins, and fats as building blocks, are typical self-assembled structures. Self-assembling phenomena exhibit different characteristics from general chemical reactions in terms of reaction progression and interaction. Self-assembling phenomena have different characteristics from chemical reactions such as precipitation. In general, chemical reactions proceed in the direction of increasing disorder, but in the case of self-assembly, the reactions occurs spontaneously in a standardized form in a specific direction. In addition, self-assembly has the characteristic of forming a stable structure not by strong bonds such as covalent bonds, but by collective action of relatively weak bonds such as hydrogen bonds, ionic bonds, and van der Waals bonds. In addition, typical examples of self-assembled structure include complexes having various forms, such as block copolymers, DNA-based structures, lipid bilayers, and colloids.

Hybridization or hybrid probes, used in molecular biology, are DNA or RNA fragments of diverse lengths (usually 20-100 base pairs in length) that can be radioactively or fluorescently labeled, and can be used in DNA- or RNA-containing samples to detect the presence of a nucleotide material (target nucleic acid) complementary to the sequences of the probes. Accordingly, the probe hybridizes with a single-stranded nucleic acid (DNA or RNA) having a base sequence allowing probe-target base pairs due to complementarity between the probe and the target nucleic acid. Generally, it is first denatured into single-stranded DNA (ssDNA) (under alkaline conditions, such as heating or exposure to sodium hydroxide), and then the labeled probe hybridizes with a target ssDNA (Southern blotting) or RNA (northern blotting) immobilized on a membrane or in situ. To detect hybridization of a probe with a target sequence, the probe is tagged (or “labeled”) with a molecular marker of either a radioactive molecule or a fluorescent molecule. Commonly used markers are ³²P (a radioactive isotope of phosphorus incorporated into the phosphodiester bond of the probe DNA) or are digoxigenin, which is a non-radioactive antibody-based marker. DNA sequences or RNA transcripts with medium-level to high-level sequence similarity to the probe are detected by visualizing the hybridized probe via autoradiography or other imaging techniques. Typically, an X-ray image of a filter is taken, or a filter is placed under an ultraviolet light. Detection of sequences with medium-level or high-level similarity depends on how stringent the hybridization conditions are. Highly stringent conditions, such as high hybridization temperature and low salinity of the hybridization buffer, allow only hybridization between very similar nucleic acid sequences, whereas less stringent conditions, such as low temperature and high salinity, allow hybridization between nucleic acid sequences that are less similar. Hybridization probes used in DNA microarrays refer to DNA covalently bonded to an inert surface, such as a coated glass slide or gene chip, to which a mobile cDNA target hybridizes.

Raman spectroscopy is spectroscopy that measures the molecular-specific scattering frequency using the Raman effect, which is a phenomenon in which when molecules are irradiated with short-wavelength light such as a laser beam, a portion of the incident light changes the polarizability of the molecules, whereby the frequency of the changed polarizability resonates with the inherent scattering frequency of the molecules. Raman spectroscopy is a method of directly irradiating a measurement target sample with light. It is easy to measure, it is possible to measure even a trace amount of sample, there is no interference of moisture and carbon dioxide, and it can be used in the visible region. Therefore, Raman spectroscopy typically uses a visible laser to detect Raman scattering caused by molecules.

As shown in FIG. 7, there are three classes of scattering: Rayleigh scattering with no change in frequency between incident light and scattering light; Stokes scattering in which incident light loses energy when colliding with atoms, and the frequency thereof decreases; and Anti-Stokes scattering in which incident light gains energy when colliding with atoms, and the frequency thereof increases. Scattering of light having less or more energy than the original incident light is called Raman scattering. Vibrational energy cannot be directly measured, but can be indirectly measured by observing whether energy is lost or gained compared with Rayleigh scattering.

That is, in Raman spectroscopy, when materials are irradiated with strong light having a single wavelength, most of the materials exhibit elastic scattering, but a part of the light is used for molecular resonance and is scattered with a different frequency. This is called inelastic scattering (Raman effect) which is used to analyze the chemical composition and structure of molecules. The degree of shift caused by Raman compared to elastic scattering is called a Raman shift, and this can be expressed as a spectrum to indicate the characteristics of a medium (FIG. 9).

By using Raman spectroscopy, signals can be obtained even in the case of non-polar molecules that change in an induced polarization rate. In fact, almost all organic molecules have an inherent Raman shift (cm⁻¹). Since the wavelength of the Raman emission spectrum indicates the chemical composition and structural characteristics of light-absorbing molecules in a sample, the analyte in the sample can be directly identified by analyzing a Raman scattering signal.

In Raman spectroscopy, a sample pretreatment process is simple, measurement can be performed easily and quickly even with a trace amount of sample, and real-time analysis is enabled because a measurement time is short (1 to 60 seconds). In addition, there is no interference of moisture and carbon dioxide, and it can be used in the visible light region. Therefore, Raman spectroscopy typically uses a visible laser to detect Raman scattering from molecules. In addition, since it is not interfered by water molecules, it is suitable for detection of biomolecules such as lipids, proteins, and genes. In addition, since there is little interference of water during measurement, it is possible to measure liquid samples such as samples (blood, urine, mucus, mucus, etc.) extracted from animals (including humans), culture media, or environmental sample extracts.

By Raman spectroscopy, cellular constituents such as proteins, lipids, and nucleic acids can be analyzed. In the Raman shift range of 400 to 3200 cm⁻¹, different signal intensities depending on the characteristics of constituent substances can be expressed as a result called Raman spectra. Typically, laser beams with wavelengths (for example, 532 nm, 633 nm, 785 nm) that have the least background effect caused by fluorescence are used.

The Raman shift information of chemical bonds and cellular constituent substances exhibiting resonance with the 532-nm laser beam are shown in Table 1.

TABLE 1
Assignments of the Raman vibrational bands in bacterial cells

| Frequency | | |
|---|---|---|
| (cm⁻¹) | Assignment | Reference |
| 520-540 | S—S str | Maquelin et al. (2002) |
| 540 | COC glycosidic ring def | Maquelin et al. (2002) |
| 665 | Guanine | Maquelin et al. (2002) |
| 720 | Adenine | Maquelin et al. (2002) |
| 785 | Cytosine, uracil (ring, str) | Maquelin et al. (2002) |
| 838 | DNA | Deng et al. (1999) |
| 858 | CC str, COC 1,4 | Maquelin et al. (2002) |
| | glycosidic link | |
| 897 | COC str | Maquelin et al. (2002) |
| 1004 | Phenylalanine | Maquelin et al. (2002) |
| 1061 | C—N and C—C str | Maquelin et al. (2002) |
| 1085 | C—O str | Maquelin et al. (2002) |
| 1102 | >PO₂⁻ str (sym) | Maquelin et al. (2002) |
| 1129 | C—N and C—C str | Maquelin et al. (2002) |
| 1295 | CH₂ def | Maquelin et al. (2002) |
| 1440-1460 | C—H₂ def | Maquelin et al. (2002) |
| 1575 | Guanine, adenine | Maquelin et al. (2002) |
| | (ring stretching) | |
| 1606 | Phenylalanine | Maquelin et al. (2002) |
| 1614 | Tyrosine | Maquelin et al. (2002) |
| 1735 | >C═O ester str | Maquelin et al. (2002) |
| 2935 | str CH₂ Asymmetric | Williams and Edwards (1994) |
| 3060 | str CH Olefinic | Williams and Edwards (1994) |

str, stretching;
def, deformation;
sym, symmetric;
asym, antisymmetric

In addition, chemical bonds such as C—C and C—N, and characteristics of various constituent substances such as DNA and amino acids can be identified.

However, despite the advantage of being able to directly identify the analyte, since the signal strength of a Raman scattering signal is very weak, there are disadvantages in that expensive equipment for detection is required, and signal reproducibility is low. Surface-enhanced Raman scattering has been reported as one of the methods to overcome these problems.

Au and Ag are very stable because they have a higher density of free electrons than other metals and have a relatively low ionization tendency. In addition, the high density of free electrons makes the real part of the permittivity of the metal negative and makes the metal have a large polarization rate, thereby inducing strong electric field enhancement. In addition, since the imaginary part indicates the degree of light absorption, which is an energy loss, the value needs to be small for efficient enhancement.

Therefore, in the case of Au, at about 630 nm in the visible region, the real part of the permittivity has a relatively small value, and the imaginary part has the lowest value. In the case of Ag, when both the real part and the imaginary part of the permittivity are considered, effective enhancement occurs at about 530 nm.

Surface plasmon (see FIG. 8) refers to a collective oscillation phenomenon of free electrons propagating along an interface between a metal and a dielectric material, in which the metal (for example, Ag or Au) has a permittivity less than 0, and the dielectric material has a permittivity greater than 0 to which the metal belongs. Here, surface plasmon resonance (SPR) refers to a phenomenon in which the frequency of an electromagnetic field (usually visible light) incident on a metal object is identical to the frequency of a surface plasmon, resulting in resonance which results in a magnitude greater than that of the incident wave. The SPR has the form of an evanescent wave that exponentially decreases with an increasing distance from the interface. The SPRs include (a) propagation-type plasmon occurring on a thin metal plane and (b) localized surface plasmon resonance (LSPR) occurring on metal nanoparticles (FIG. 8).

Surface enhanced Raman spectroscopy (SERS) uses the principle that light incident on the surface of metal nanoparticles such as silver or gold nanoparticles causes plasmon resonance on the surface, thereby amplifying a Raman scattering signal. That is, a phenomenon in which when a target molecule exists around a metal nanostructure, the Raman scattering signal of the molecule greatly increases is used. One of the advantages of surface-enhanced Raman scattering analysis is that information that is difficult to obtain with general Raman analysis can be obtained.

Surface-enhanced Raman spectroscopy (SERS) can be used to amplify the Raman scattering signal that is relatively hard to detect due to a small Raman scattering cross-sectional area. By using metal nanoparticles such as silver (Ag) or gold (Au), Raman scattering signals of molecules adsorbed on the surface of the nanoparticles can be amplified and detected by the interaction between the metal nanoparticle and the incident light.

In this case, the degree of amplification of the signal varies depending on the shape and size of metal nanoparticle and the type of metal, and is also affected by the angle, wavelength, and polarization of incident light. When these factors are controllable, the Raman scattering signal of a single molecule can be analyzed by SERS.

In the case of surface-enhanced Raman spectroscopy, the low detection intensity and the ability to detect the signal even with a trace amount of sample are very attractive for biosensor applications. In addition, unlike conventional fluorescence analysis technology, information on the chemical structure of a sample is provided as a narrow spectrum. In addition, since each molecule has a unique Raman scattering signal (Raman shift value), multiple detection is possible at the same time. Many studies on detecting biomaterials (DNA, proteins, cells, etc.) and on implementing disease diagnoses, utilizing the characteristics of the surface-enhanced Raman spectroscopy, have been reported.

Dielectric detection methods mostly use fluorescence signals or fluorescence images. Since fluorescence images and signals are identified by color and signal intensity, there is a limit to the increase in signal intensity unless the fluorescent material is present at a certain concentration or higher. In addition, even though the types of fluorescent materials are different, since there is a limit (3 to 5 types) in the number of colors that can be expressed, there are many restrictions in differentiating detection materials. Due to the limitations of such fluorescence signals, a material amplification method is used to detect nano-sized dielectrics as fluorescence signals. Most of the equipment (NGS, microarrays, etc.) that detects these target fluorescence signals are very expensive and delicate and require specialized operation skills.

RT-PCR, which is a technology mainly used for virus detection, is relatively inexpensive, but it is generally used for genome amplification. It requires stringent detection conditions (for example, non-target substances may be amplified when contamination occurs even in trace amounts, causing errors in test results), it is time consuming (approximately 2 to 3 hours), and it requires experimental proficiency (even among clinical pathologists, if the proficiency is not high, an error occurs in the test result). In the case of RT-PCR, the accuracy is higher than that of antigen and antibody tests, but it is more complicated and takes a long time, such as requiring supplementation such as conducting genetic tests more than twice, training on sample collection methods, and comparative evaluation of reagents.

Therefore, as a genome molecular diagnosis method, a technology which is an on-site simple diagnosis technology and requires quantitative detection, unlike RT-PCR, is needed. Therefore, the development of in vitro molecular diagnosis technology using Raman scattering signals is urgently needed.

On the other hand, research is being actively conducted to perform early detection of genes and proteins (biomarkers) related to various diseases, using high-sensitivity DNA analysis and SERS sensors. Unlike other analysis methods (infrared spectroscopy), Raman spectroscopy has several advantages. Infrared spectroscopy can obtain a strong signal in the case of molecules with a dipole moment change, while Raman spectroscopy can obtain a strong signal in the case of non-polar molecules with an induced polarization change. Therefore, almost all organic molecules have an inherent Raman shift (cm⁻¹). In addition, since it is not affected by interference of water molecules, it is suitable for detection of biomolecules such as lipids, proteins, and genes.

As described above, the Raman scattering signal is the most suitable optical signal for detecting each type of molecule. However, since the Raman scattering signal is unstable and weak in a liquid phase, a nanostructure capable of amplifying the signal is required. In addition, existing methods used for Raman scattering signal amplification include Surface Enhanced Raman Scattering (SERS) and Tip Enhanced Raman Scattering (TERS). However, signal amplification is mainly performed on dry samples, it has a disadvantage that it cannot be stably applied to liquid samples.

On the other hand, since biosensors are related to human health, quality of daily lives, and life, efforts to increase accuracy as well as to increase sensor sensitivity are required. When a sensor has high sensitivity to a specific signal, the sensitivity to non-specific signals is also high. Therefore, there may be cases in which incorrect results are transmitted. This can lead to side effects such as abuse of drugs and unnecessary treatment. Therefore, this should be considered first when developing sensors.

### DISCLOSURE

### Technical Problem

The present invention aims to achieve (1) solving the problem that when the sensitivity of a sensor increases, the sensitivity to non-specific signals increases at the same time, (2) designing around the difficulty of synthesizing and controlling precisely structurally defined nanomaterials, (3) forming a nanostructure in which an optical signal can be enhanced through localized surface plasmon resonance (LSPR); and (4) securing a reproducible optical signal derived from the nanostructure.

To this end, the present invention is intended to design, as a main component of a target nucleic acid detecting agent, a nucleic acid based self-assembled complex capable of increasing the resolution of quantification of a target nucleic acid target by enhancing the signal to noise ratio (S/N ratio) depending on the presence or absence of the target nucleic acid.

In addition, the present invention aims to design a nucleic acid based self-assembled complex that is structurally stable in a liquid sample so as to reproducibly amplify an optical signal such as a Raman scattering signal, which is unstable and weak in a liquid phase when irradiated with light.

Specifically, the present invention is intended to provide a nucleic acid based self-assembled complex for Raman detection of a target nucleic acid. To this end, a novel nucleic acid based self-assembled complex is designed by using only pre-synthesized metal nanoparticles so that when forming nanogaps on metal nanoparticle-based structures for generating and further enhancing a surface plasmon resonance phenomenon (electromagnetic effect), uniform nanogaps are reproducibly provided at regular intervals of several nanometers by metal nanoparticles. Thus, the nanogaps, each structurally accurately and precisely defined between every two metal nanoparticles, are formed, and Raman indicators are interposed in the nanogaps that cause localized surface plasmon resonance (LSPR) that amplifies a Raman scattering signal. Therefore, the nucleic acid based self-assembled complex can serve as an on/off signal system sensor by which whether the nanogaps are formed or not is determined by an inverse functional relation with the presence or absence of a target nucleic acid to be measured in order to secure a reproducible enhanced Raman scattering signal, and by which the target nucleic acid can be quantified.

In surface-enhanced Raman spectroscopy (SERS), the degree of amplification of the Raman scattering signal has many problems to be solved in terms of reproducibility and reliability due to change in enhancement efficiency according to the wavelength of light used when measuring the spectrum and to the direction of polarization. Thus, a nucleic acid based self-assembled complex to solve the problems is newly designed, and it is investigated whether the nucleic acid based self-assembled complex can serve as a target nucleic acid detecting sensor. As a result, it has been found that the nucleic acid based self-assembled complex has structural stability so as to produce reproducible scattering light signals even when it is in Brownian motion in liquid, and the present invention has been made based on the findings.

### Technical Solution

A first aspect of the present invention provides: A nucleic acid based self-assembled complex of turn-off based way for detecting a target nucleic acid, which has a structural stability so as produce reproducible scattering light signal regardless of Brownian motion in liquid, wherein the nucleic acid based self-assembled complex comprises:
(a) a first nanoparticle based structure in which at least one first nucleotide which is a probe being hybridized with the target nucleic acid depending on a condition is linked to a first metal nanoparticle, and (b) a second nanoparticle based structure in which at least one second nucleotide which is 10 bp or more complementary to the first nucleotide is linked to a second metal nanoparticle, wherein the nucleic acid based self-assembled complex is self-assembled through complementary hydrogen bonding between the first nucleotide and the second nucleotide, wherein when the nucleic acid based self-assembled complex is formed, a nanogap is formed by adjacent first metal nanoparticle and second metal nanoparticle, and
optionally, (ii) the nanogap is designed to a space which generates and further enhances a surface plasmon resonance phenomena when a light is applied to the nanogap.

A second aspect of the present invention provides: A target nucleic acid detecting agent which detects a target nucleic acid by turn-off method by using a reproducible scattering light signal derived by a nucleic acid based self-assembled complex which is in brownian motion in liquid, wherein the nucleic acid based self-assembled complex comprises:
(a) a first nanoparticle based structure in which at least one first nucleotide which is a probe being hybridized with the target nucleic acid depending on a condition is linked to a first metal nanoparticle, and (b) a second nanoparticle based structure in which at least one second nucleotide which is 10 bp or more complementary to the first nucleotide is linked to a second metal nanoparticle, wherein the nucleic acid based self-assembled complex is self-assembled through complementary hydrogen bonding between the first nucleotide and the second nucleotide, wherein the nucleic acid based self-assembled complex is designed to detect a changing scattering signal when the nucleic acid based self-assembled complex is not formed, or is disassembled, by hybridization of the first nucleotide and the target nucleic acid if the target nucleic acid exists.

A third aspect of the present invention provides: A method of detecting a target nucleic acid in the liquid by turn-off based way using reproducible scattering light signal derived by a nucleic acid based self-assembled complex which is in brownian motion in liquid, comprising:
a first step, which is preparing a target nucleic acid detecting agent being designed to, detect a changing scattering signal when a nucleic acid based self-assembled complex is not formed, or is disassembled, by hybridization of the first nucleotide and the target nucleic acid if the target nucleic acid exists, wherein the nucleic acid based self-assembled complex comprises: (a) a first nanoparticle based structure in which at least one first nucleotide which is a probe being hybridized with the target nucleic acid depending on a condition is linked to a first metal nanoparticle, and (b) a second nanoparticle based structure in which at least one second nucleotide which is 10 bp or more complementary to the first nucleotide is linked to a second metal nanoparticle, wherein the nucleic acid based self-assembled complex is self-assembled through complementary hydrogen bonding between the first nucleotide and the second nucleotide;
a second step which is performing hybridization reaction in a nucleic acid containing liquid sample, with a target nucleic acid detecting agent of the first step, containing (a) the first nanoparticle based structure in which the first nucleotide is linked to the first metal nanoparticle, and (b) the second nanoparticle based structure in which the second nucleotide is linked to the second nanoparticle;
a third step which is measuring optical signal derived from the nucleic acid based self-assembled complex, before and after, and/or simultaneously the hybridization reaction of the second step; and
a fourth step which is providing a detection, and/or quantitative data of the target nucleic acid in the sample, through algorithm of which analyzes the optical signal or a change thereof measured in the third step.

Hereinafter, the present invention will be described.

The present invention is characterized by providing a nucleic acid based self-assembled complex of turn-off based way for detecting a target nucleic acid, which has a structural stability so as produce a reproducible scattering light signal regardless of Brownian motion in liquid. To this end, according to the present invention, a nucleic acid based self-assembled complex that is self-assembled through complementary hydrogen bonding of at least 10 base pairs (bp) is designed, and the nucleic acid based self-assembled complex can continuously provide reproducible optical signals during measurement due to structural stability thereof regardless of being in Brownian motion in liquid.

The nucleic acid based self-assembled complex of the present invention includes: (a) a first nanoparticle based structure in which at least one first nucleotide, which is a probe hybridizing with a target nucleic acid depending on a condition, is linked to a first metal nanoparticle; and (b) a second nanoparticle based structure in which at least one second nucleotide that is 10 bp or more complementary to the first nucleotide is linked to a second metal nanoparticle, in which the nucleic acid based self-assembled complex is self-assembled through complementary hydrogen bonding between the first nucleotide and the second nucleotide.

When the nucleic acid based self-assembled complex is formed, (i) a nanogap is defined by the first and second metal nanoparticles adjacent to each other, and (ii) the nanogap is designed to serve as a space that generates and further enhances a surface plasmon resonance phenomenon when irradiated with light.

Among the nucleic acid based self-assembled complexes illustrated in FIGS. 1 and 2, the NEW structure is designed such that it includes (a) a first nanoparticle-based structure in which a first nucleotide, which is a probe hybridizing with a target nucleic acid, is linked to a first metal nanoparticle (b) a second nanoparticle-based structure in which a second nucleotide that is at least 10 bp complementary to the first nucleotide is linked to a second metal nanoparticle, and is self-assembled through complementary hydrogen bonding between the first nucleotide and the second nucleotide. In this case, (i) a nanogap is formed by the first and second metal nanoparticles adjacent to each other, (ii) the nanogap serves as a space that causes a surface plasmon resonance phenomenon (electromagnetic effect) when irradiated with light, and (iii) a Raman indicator linked to the second nucleotide is interposed in the nanogap to enhance a Raman scattering signal to be detected when irradiated with light.

Surprisingly, it has been observed through a video that through nanoparticle tracking analysis (NTA) that measures and visualizes particle sizes and concentrations by capturing light scattered from particles being in Brownian motion, the concentration of the above-described nucleic acid based self-assembled structures (NEW structure) in a target nucleic acid detecting agent can be measured, and the two adjacent metal nanoparticles in the above-described nucleic acid based self-assembled complex (NEW structure) perform Brownian motion (FIG. 4). A nanoparticle tracking analyzer (NTA) used through the observation is a device that (1) provides high-resolution particle size information on the nanoparticle sample after nanoparticle tracking analysis using particle-by-particle light scattering, (2) records the velocity or spread of the nanoparticles in a solution at a light scattering point, (3) identifies each light scattering point of each particle by software so as to be included in a data set, and (4) uses a phenomenon in which smaller nanoparticles move faster than larger nanoparticles due to Brownian motion and scatter a less amount of light, and (5) enables visual confirmation and result verification by collecting light scattered from the nanoparticles to visually determine the presence.

The fact that two adjacent metal nanoparticles perform Brownian motion was confirmed through a video, and the fact that the concentration of nucleic acid based self-assembled complexes (NEW structures) in a target nucleic acid detecting agent can be measured was confirmed through the nanoparticle tracking analysis (NTA). The facts are based on the premise that the formation of dimer other than trimer or higher multimer is controlled in terms of metal nanoparticles, in the case of a nucleic acid based self-assembled complex (NEW structure) that includes (a) a first nanoparticle-based structure in which a first nucleotide is linked to a first metal nanoparticle and (b) a second nanoparticle-based structure in which a second nucleotide that is at least 10 bp complementary to the first nucleotide is linked to a second metal nanoparticle, in which the self-assembly is achieved by complementary hydrogen bonding between the first nucleotide and the second nucleotide. In addition, this suggests that the nucleic acid based self-assembled complex (NEW structure) of the present invention can reproducibly and continuously provide an optical signal such as scattering light even while being in Brownian motion.

In addition, it was surprisingly found that the above-described nucleic acid based self-assembled complex (NEW structure) in the target nucleic acid detecting agent can continuously provide reproducible Raman scattering signals during measurement when irradiated with light, regardless of being in Brownian motion in liquid (FIG. 3). From this, in the case of designing the above-described nucleic acid based self-assembled complex (i) by using only uniformly pre-synthesized metal nanoparticles and (ii) being self-assembled through complementary hydrogen bonding of at least ten base pairs (10 bp), to provide reproducible uniform nanogaps arranged at regular intervals of several nanometers, the nanogaps provided by the nucleic acid based self-assembled complex (NEW structure) has structural stability so as to continuously provide a reproducible enhanced optical signal during measurement regardless of being in Brownian motion in liquid.

That is, the present inventors have found that the nucleic acid based self-assembled complex produced by spontaneous molecular-level hydrogen bonding between a first nanoparticle hybridizing with a target nucleic acid and a second nucleotide complementary to the first nucleotide, i.e., complementary hydrogen bonding of at least ten base pairs (10 bp), have a structural stability so as to continuously provide reproducible optical signals during measurement regardless of being in Brownian motion in liquid, and the present invention is based on the finding.

Accordingly, the present invention provides a nucleic acid based self-assembled complex of turn-off based way for detecting a target nucleic acid, which has a structural stability so as produce reproducible scattering light signals regardless of Brownian motion in liquid. In addition, the present invention provides a turn-off based method of detecting a target nucleic acid, using a reproducible optical signal captured by a nucleic acid based self-assembled complex being in Brownian motion in liquid, and a target nucleic acid detecting agent.

Here, the nucleic acid based self-assembled complex of turn-off based way for detecting a target nucleic acid, and the target nucleic acid detecting agent includes at least: (a) a first nanoparticle based structure in which at least one first nucleotide, which is a probe hybridizing with the target nucleic acid depending on a condition, is linked to a first metal nanoparticle; and (b) a second nanoparticle based structure in which at least one second nucleotide that is 10 bp or more complementary to the first nucleotide is linked to a second metal nanoparticle.

According to the present invention, the nucleic acid based self-assembled complex of turn-off based way for detecting a target nucleic acid, and the target nucleic acid detecting agent are designed such that when forming the nucleic acid based self-assembled complex, (i) a nanogap is formed by first and second metal nanoparticles adjacent to each other, and preferably, (ii) the nanogap is designed to serve as a space that generates and further enhances a surface plasmon resonance phenomenon when irradiated with light.

For example, the first nucleotide hybridizing with the target nucleic acid includes: (i) an oligonucleotide probe having a nucleic acid sequence that hybridizes with a part of the sequence of the target nucleic acid depending on a condition, (ii) a spacer that helps 10 bp or more complementary hydrogen bonding by increasing the moving degree of freedom, and (iii) an oligonucleotide attacher that attaches to the first metal nanoparticle;

the second nucleotide that is 10 bp or more complementary to the first nucleotide may include: (i) a 2-1 oligonucleotide attacher having a sequence complementary to the oligonucleotide probe of the first nucleotide, (ii) a spacer that helps 10 bp or more complementary hydrogen bonding by increasing the moving degree of freedom, and (iii) a 2-2 oligonucleotide attacher that attaches to the second metal nanoparticle.

Optionally, the second nucleotide is a nucleotide in which a Raman indicator of which a Raman shift value is known, is linked between (i) the 2-1 oligonucleotide attacher having a nucleic acid sequence complementary to that of the oligonucleotide probe of the first nucleotide and (ii) the spacer (FIG. 2(A)); or

the first nucleotide is a nucleotide in which a Raman indicator of which a Raman shift value is known, is linked between (i) the oligonucleotide probe having a nucleic acid sequence that hybridizes with a part of the sequence of the target nucleic acid and (ii) the spacer (FIG. 2(B)).

The oligonucleotide attacher attaching to the metal nanoparticle serves as an adhesive for attaching the first nucleotide and the second nucleotide to the first metal nanoparticle and the second metal nanoparticle, respectively. It may be poly-adenine (2 to 30 mer). For example, it is 10 mer-adenine.

When gold nanoparticles are used as the first metal nanoparticle and/or the second metal nanoparticle, the gold nanoparticles and the oligonucleotide attachers, which are organic molecules, can be stably bonded, resulting in stable performance even under conditions of high salt concentration, high temperature, and long-term storage.

The spacer serves as a bearing hinge that helps increasing the degree of freely moving of the oligonucleotide probe of the first nucleotide and the 2-1 oligonucleotide attacher to facilitate 10 bp or more complementary hybridization. To facilitate the control of length and orientation, it may be (CH₂)ₙ (n=3 to 20), for example (CH₂)₁₂.

The target nucleic acid detecting agent of the present invention may contain or form a nucleic acid based self-assembled complex of turn-off based way for detecting a target nucleic acid. For quality assurance such as concentration and storage stability, the target nucleic acid detecting agent for detecting a target nucleic acid in a turn-off way, according to the present invention, preferably contains a known concentration of the nucleic acid based self-assembled complexes.

The nucleic acid based self-assembled complex of turn-off based way for detecting a target nucleic acid, and the target nucleic acid detecting agent, according to the present invention are designed to measure a change in optical signal when the nucleic acid based self-assembled complex is not formed by hybridization of the first nucleotide with the target nucleic acid or is disassembled when the target nucleic acids exist (see FIGS. 1, 2, 5, and 6).

Therefore, when formation or non-formation of the nucleic acid based self-assembled complexes and/or the degree of formation (quantification) of the nucleic acid based self-assembled complexes can be confirmed on the basis of a reproducible optical signal captured by the nucleic acid based self-assembled complexes being in Brownian motion, by using the target nucleic acid detecting agent, it is possible to detect or quantify the target nucleic acids hybridizing the first nucleotides so that the nucleic acid based self-assembled complexes are not formed, or are disassembled.

The number of nucleic acid based self-assembled complexes formed by spontaneous molecular-level hydrogen bonding between the first nucleotide, which is a probe that hybridizes with the target nucleic acid, and the second nucleotide complementary to the first nucleotide, has an inverse functional relation with respect to the number of target nucleic acids (FIGS. 1, 5, and 6). That is, (i) when the target nucleic acids in the sample are absent or present in an amount that is below the lower limit of the detection sensitivity of the target nucleic acid detecting agent for the target nucleic acids (the minimum value of the detection range of the target nucleic acid detecting agent containing or forming nucleic acid based self-assembled complexes), the number of nucleic acid based self-assembled complexes formed by self-assembly of the first and second nucleotides that are complementary to each other is maximum, and (ii) when the target nucleic acids are present in an amount that is higher than the upper limit of the detection sensitivity of the target nucleic acid detecting agent for the target nucleic acids, the number of nucleic acid based self-assembled complexes formed by self-assembly of the first and second nucleotides that are complementary to each other is minimum (FIG. 5).

In this case, when an indicator that exhibits an already-known optical signal is linked to the second oligonucleotide that competes with the target nucleic acid for hybridization with the first nucleotide, the formation or non-formation, the number, and the concentration of the nucleic acid based self-assembled complexes can be confirmed (quantified) on the basis of the optical signal of the indicator (FIGS. 1 and 6). In addition, when the second oligonucleotide competing with the target nucleic acid for hybridization with the first nucleotide linked to the nanoparticle is linked to another nanoparticle, the formation or non-formation/number/concentration of the nucleic acid based self-assembled complexes in the target nucleic acid detecting reagent can be measured through nanoparticle tracking analysis (NTA) that measures and visualizes particle sizes and concentrations by capturing light scattered from particles being in Brownian motion (FIG. 2(C) and FIG. 3).

In addition, when designing a nucleic acid based self-assembled complex that is self-assembled to form a stable and regular structure through complementary hydrogen bonding between the first nucleotide and the second nucleotide, which are complementary to each other, the present invention is characterized by linking at least one first nucleotide, which is a probe that hybridizes with the target nucleic acid, and at least one second nucleotide complementary to the first metal nanoparticle, to the first nucleotide and the second metal nanoparticle, respectively, so that the nucleic acid based self-assembled complex can be in Brownian motion.

In the present specification, although the first metal nanoparticle and the second metal nanoparticle are replaced with non-metal particles, such a modification falls within the scope of the present invention as long as the particles can exhibit scattering light signals while being in Brownian motion in liquid.

That is, the present invention designs and provides, as a self-assembled unit that has a stable and regular structure by spontaneous molecular-level non-covalent bonding,
(a) a first nanoparticle based structure in which at least one first nucleotide, which is a probe hybridizing with the target nucleic acid depending on a condition, is linked to a first nanoparticle, and (b) a second nanoparticle based structure in which at least one second nucleotide that is 10 bp or more complementary to the first nucleotide is linked to a second nanoparticle.

In this case, when the first nanoparticle to which the first nucleotide that hybridizes with the target nucleic acid is linked and/or the second nanoparticle to which the second nucleotide complementary to the first nucleotide is linked are metal nanoparticles that exhibit a localized surface plasmon resonance (LSPR) phenomenon, the light signal can be amplified through strong electromagnetic field enhancement by inducing plasmon resonance on the surface of the metal nanoparticles upon light irradiation.

Therefore, when the nucleic acid based self-assembled complex is formed, (iii) if the nucleic acid based self-assembled complex is designed such that an indicator emitting a known optical signal is interposed within a distance of 3 nm or less to the metal nanoparticle when the indicator is linked to the first or second nucleotide, the optical signal emitted from the indicator when the nucleic acid based self-assembled complex is formed is amplified, and thus the resolution of quantification of the target nucleic acid is improved (FIG. 6). For example, since the target nucleic acid quantification range can be set for each target nucleic acid detecting agent by controlling the maximum concentration of the nucleic acid based self-assembled complexes that can be formed in the target nucleic acid detecting agent (e.g., 10¹ pcs/ml, 10² pcs/ml, 10³ pcs/ml, 10⁴ pcs/ml, etc.), a customized quantitative threshold can be applied for diagnosis.

Furthermore, the present invention features that it is possible to determine the formation or non-formation and/or degree of formation (quantification) of the nucleic acid based self-assembled complexes on the basis of the optical signals of the nucleic acid based self-assembled complexes being in Brownian motion.

To this end, the nucleic acid based self-assembled complex of the present invention may be a nucleic acid based self-assembled complex formed by complementary hydrogen bonding between a first nucleotide and a second nucleotide, in which the nucleic acid based self-assembled complex includes (a) a first nanoparticle based structure in which at least one first nucleotide, which is a probe hybridizing with a target nucleic acid depending on a condition, is linked to a first metal nanoparticle and (b) a second nanoparticle based structure in which at least one second nucleotide that is 10 bp or more complementary to the first nucleotide is linked to a second metal nanoparticle.

Since the first nanoparticle based structure in which the first nucleotide is linked to a nanoparticle and the second nanoparticle based structure in which the second nucleotide is linked to a nanoparticle form a nucleic acid based self-assembled complex performing Brownian motion in liquid through complementary hydrogen bonding between the first nucleotide and the second nucleotide, (1) the concentration of the aforementioned nucleic acid based self-assembled complexes in liquid can be measured through nanoparticle tracking analysis (NTA) that measures and visualizes particle sizes and concentrations by capturing light scattered from the particles being in Brownian motion, and (2) since the second nucleotide competes with the target nucleic acid for hybridization with the first nucleotide probe, the number of the nucleic acid based self-assembled complexes has an inverse functional relation with the number of target nucleic acids. That is, the target nucleic acids can be quantified through nanoparticle tracking analysis (NTA).

In addition, the nucleic acid based self-assembled complex produced by spontaneous molecular-level hydrogen bonding between (a) a first nanoparticle based structure in which at least one first nucleotide, which is a probe hybridizing with the target nucleic acid depending on a condition is linked to a first metal nanoparticle, and (b) a second nanoparticle based structure in which at least one second nucleotide which is 10 bp or more complementary to the first nucleotide is linked to a second metal nanoparticle, can form a nanogap having a structural stability so as to continuously provide reproducible optical signals during measurement regardless being in Brownian motion in liquid. Therefore, when the nucleic acid based self-assembled complex is designed such that (iii) a Raman indicator linked to the first nucleotide or the second nucleotide is interposed in the nanogap so that a Raman scattering signal derived from the Raman indicator is enhanced when irradiated with light, formation or non-formation/number/concentration of the nucleic acid based self-assembled complexes can be determined (quantified) by enhancing the signal to noise ratio (S/N ratio), and the resolution of quantification of the target nucleic acids can be increased when determining (quantifying) the presence or absence/number/concentration of the target nucleic acids.

For example, as shown in FIG. 1, when forming nanogaps on the nucleic acid based self-assembly complexes to which metal nanoparticles are applied to generate and further enhance a surface plasmon resonance phenomenon (electromagnetic effect), only uniform pre-synthesized metal nanoparticles are used to reproducibly provide uniform nanogaps that are arranged at regular intervals of several nanometers by the metal nanoparticles, so that each nanogap that is precisely structurally defined is formed between every two metal nanoparticles, and Raman indicators are disposed in the respective nanogaps that cause a localized surface plasmon resonance (LSPR) phenomenon that amplifies the Raman scattering signal. Therefore, the formation or non-formation/number of the nanogaps can be determined by an inverse functional relation with the presence or absence of the target nucleic acids, and the nucleic acid based self-assembled complex can serve as a turn-off signal system sensor when the target nucleic acid exists. Therefore, the target nucleic acids can be quantitatively analyzed. In conclusion, the nanogaps formed by the nucleic acid based self-assembled complexes of the present invention can solve the problems of the Raman scattering signals that are weak in intensity and low in signal reproducibility when quantifying the target nucleic acids.

The nucleic acid base self-assembled complex formed by a self-assembly of (a) a first nanoparticle based structure in which at least one first nucleotide is linked to a first metal nanoparticle, which is a probe that hybridizes with a target nucleic acid according to conditions, and (b) a second nanoparticle based structure in which at least one second nucleotide which is 10 bp or more complementary to the first nucleotide is linked to a second metal nanoparticle, serves as a turn-off signal system sensor when the target nucleic acid exists. Therefore, a target nucleic acid detecting agent that contains or forms a known concentration of nucleic acid based self-assembled complexes has a characteristic of securing or estimating reference points (Min and Max) of an optical signal for each concentration of the nucleic acid based self-assembled complexes in the target nucleic acid detecting agent on the basis of the fact that the intensity of the optical signal of the Raman indicator is strongest when the target nucleic acid does not exist, the intensity of the optical signal of the Raman indicator gradually decreases according to increasing of an amount of the target nucleic acids, and the intensity of the Raman signal is weakest when the target nucleic acids corresponding to the known concentration of the nucleic acid based self-assembled complexes exists at an excessive amount (FIG. 5).

Accordingly, as long as the nucleic acid based self-assembled complex is formed by self-assembly of (a) a first nanoparticle based structure in which at least one first nucleotide, which is a probe hybridizing with a target nucleic acid depending on a condition, is linked to a first metal nanoparticle and (b) a second nanoparticle based structure in which at least one second nucleotide that is 10 bp or more complementary to the first nucleotide is linked to a second metal nanoparticle, an improved nucleic acid based self-assembled complex capable of detecting or quantifying the target nucleic acid in a turn-off way also falls within the scope of the present invention.

[Target Nucleic Acid]

When the nucleic acid based self-assembled complex of the present invention is applied to a target nucleic acid detecting agent, the target nucleic acid can be selectively and specifically detected in a turn-off way through complementary hydrogen bonding between the first nucleotide, which is a probe that hybridizes with the target nucleic acid, and the target nucleic acid.

Non-limiting examples of target nucleic acids that can be detected using the nucleic acid based self-assembled complex of the present invention as a target nucleic acid detecting reagent include genes, viral RNA and DNA, bacterial DNA, fungal DNA, mammalian DNA, cDNA, mRNA, RNA and DNA fragments, oligonucleotides, synthetic oligonucleotides, modified oligonucleotides, single-stranded and double-stranded nucleic acids, natural and synthetic nucleic acids, and the like.

In the present invention, when the target nucleic acid is a genome or a fragment thereof, quantitative and/or qualitative analysis of the genome can be performed. In addition, identification of viruses and/or microorganisms or diagnosis of diseases can be performed through the detection of target nucleic acids.

[Method of Detecting a Target Nucleic Acid in a Turn-Off Way Using a Reproducible Optical Signal Derived from a Nucleic Acid Based Self-Assembled Complex being in Brownian Motion in Liquid]

The method of detecting a target nucleic acid in a turn-off way by using a reproducible optical signal derived from a nucleic acid based self-assembled complex which is in Brownian motion in liquid, according to the present invention, includes:
a first step of preparing a target nucleic acid detecting agent that is designed to detect a change in an optical signal when a nucleic acid based self-assembled complex is not formed or is disassembled, by hybridization of the first nucleotide and the target nucleic acid when the target nucleic acid exists, in which the nucleic acid based self-assembled complex is formed by self-assembly of (a) a first nanoparticle based structure in which at least one first nucleotide which is a probe hybridizing with the target nucleic acid depending on a condition is linked to a first metal nanoparticle, and (b) a second nanoparticle based structure in which at least one second nucleotide which is 10 bp or more complementary to the first nucleotide is linked to a second metal nanoparticle;
a second step of causing a hybridization reaction with the target nucleic acid detecting agent of the first step, which contains (a) the first nanoparticle based structure in which the first nucleotide is linked to the first metal nanoparticle, and (b) the second nanoparticle based structure in which the second nucleotide is linked to the second nanoparticle, in a nucleic acid-containing liquid sample;
a third step of measuring an optical signal derived from the nucleic acid based self-assembled complex being in Brownian motion in a liquid sample before, after, and/or simultaneously with the hybridization reaction of the second step; and
a fourth step of providing detection and/or quantitative data of the target nucleic acid contained in the sample, through an algorithm that analyzes the optical signal or a change value thereof measured in the third step.

In this case, the first and second steps may be performed at a salt concentration that does not cause agglomeration of the metal nanoparticles to which negatively charged nucleotides are linked, or may use a nanoparticle based structure in which nucleotides are linked to metal nanoparticles at a minimum level of density sufficient to provide colloidal stability for dispersion of the metal nanoparticles.

The nucleic acid based self-assembled complex formed in the target nucleic acid detecting agent of the present invention may be designed to maximize the signal/noise ratio depending on the presence or absence of the target nucleic acid. For example, a second nucleotide that is 10 bp or more complementary to the first nucleotide may be labeled with a magnetic or metal nanoparticle and/or a Raman indicator.

The second step of causing a hybridization reaction with a target nucleic acid detecting agent in a nucleic acid-containing liquid sample may be performed by heating to a temperature which a temperature-dependent spontaneous non-covalent bond of the nucleic acid based self-assembled complex is released, and subsequent cooling to temperatures at which a temperature-dependent spontaneous non-covalent bond of the nucleic acid based self-assembled complex is formed, respectively.

When the target nucleic acid detecting agent of the first step contains or is capable of forming the nucleic acid based self-assembled complex of the present invention, in the second step of causing a hybridization reaction with the target nucleic acid detecting agent of the first step in the nucleic acid-containing liquid sample, the agent is heated to a temperature at which the complementary hydrogen bond between the first nucleotide and the second nucleotide is released so that the nucleic acid based self-assembled complex is disassembled, and then the agent is cooled to a temperature at which the second nucleotide and the target nucleic acid compete for hybridization with the first nucleotide probe. In this case, the target nucleic hybridizes with the first nucleotide instead of the second nucleotide, so that the nucleic acid based self-assembled complex is not re-formed (FIG. 1).

As described above, the method of detecting a target nucleic acid in a turn-off way using a reproducible optical signal derived from a nucleic acid based self-assembled complex being in Brownian motion in liquid is a method of detecting a marker and an optical signal as well as a hybridization reaction in the liquid in which particles perform Brownian motion.

The target nucleic acid detecting agent of the present invention is configured such that a first nanoparticle based structure in which a first nucleotide is linked to a nanoparticle and a second nanoparticle based structure in which a second nucleotide is linked to a nanoparticle form a nucleic acid based self-assembled complex being in Brownian motion in liquid by complementary hydrogen bonding between the first nucleotide and the second nucleotide.

The size of a nanoparticle emitting an optical signal such as a scattering light signal while being in Brownian motion in liquid is in a range of 20 nm to 500 nm.

The optical signal measured by the method of detecting a target nucleic acid in liquid, according to the present invention, may be, for example, an elastic scattering signal or an inelastic Raman scattering signal.

The target nucleic acid detecting agent used in the method of detecting a target nucleic acid in liquid, according to the present invention, is designed such that a change in an optical signal can be measured, when the nucleic acid based self-assembled complex is not formed or is disassembled by hybridization of the first nucleotide and the target nucleic acid when the target nucleic acid exists.

The nucleic acid based self-assembled complex used in the method of detecting a target nucleic acid in liquid, according to the present invention, is designed to be self-assembled through complementary hydrogen bonding of at least ten base pairs (10 bp) so as to have structural stability capable of continuously providing reproducible optical signals during measurement even while being in Brownian motion in liquid.

The term “base pair” refers to a pair of two bases capable of making hydrogen bonding, among the bases constituting a nucleic acid. In the case of Watson-Crick DNA base pairs, there are base pairs: an adenine (A) and thymine (T) pair, and a guanine (G) and cytosine (C) pair. In the case of RNA, thymine is replaced by uracil (U). Aside from the base pairs, Wobble base pairs and Hoogsteen base pairs also exist. One base pair (1 bp) corresponds to about 3.4 Å.

The sequence length of the first nucleotide is preferably longer than the sequence length of the second nucleotide so that the nucleic acid based self-assembled complex is not formed or is disassembled by hybridization of the first nucleotide with the target nucleic acid when the target nucleic acid exists.

Since the complementary hydrogen bonding between the nucleotides is an exothermic reaction, in order for the hybridization of the first nucleotide probe with the target nucleic acid to be predominant compared to the hybridization of the first nucleotide probe with the second nucleotide in a thermodynamic sense when the second nucleotide and the target nucleic acid competes for hybridization with the first nucleotide, the sequence of the first nucleotide probe that is complementary to the sequence of the target nucleic acid is preferably longer than the sequence of the second oligonucleotide complementary to the first nucleotide. In addition, when the entire genome is fragmented to be used as a target nucleic acid, the sequence length of the target nucleic acid is preferably longer than the sequence length of the second oligonucleotide.

The sequence length of the first nucleotide that hybridizes with the target nucleic acid and the sequence length of the second nucleotide that is complementary to the first nucleotide are in a range in which self-folding does not occur.

The sequence length of the first nucleotide is preferably 12 to 40 bases, and the sequence length of the second nucleotide which is complementary to the first nucleotide is preferably 10 to 40 bases.

It is preferred to modify the nanoparticles with negatively charged nucleotides of a minimum density that is sufficient to provide colloidal stability in the target nucleic acid detecting agent so that the optical signal derived from the nucleic acid based self-assembled complex being in Brownian motion in liquid becomes a particle-by-particle optical signal.

According to the present invention, the target nucleic acid detecting agent or the buffer condition of the hybridization reaction is adjusted while allowing the nucleotides to be linked to the nanoparticles at a minimum density sufficient to provide colloidal stability in the target nucleic acid detecting agent, so that a dimer-type nucleic acid based self-assembled complex rather than a higher multimer-type can be formed by self-assembly of (a) a first nanoparticle based structure in which a first nucleotide is linked to a first metal nanoparticle and (b) a second nanoparticle based structure in which a second nucleotide complementary to the first nucleotide is linked to a second metal nanoparticle, whereby the particle-by-particle optical signals have one to one correspondence, in terms of number, with the nucleic acid based self-assembled complexes being in Brownian motion in liquid.

The present invention features that it is possible to determine formation or non-formation/degree of formation (quantification) of the nucleic acid based self-assembled complexes, using the optical signals derived from the nucleic acid based self-assembled complexes, and it is possible to detect or quantify the target nucleic acids that hybridize with the first nucleotides such that the nucleic acid based self-assembled complexes are not formed or are disassembled.

For example, when the target nucleic acid detecting agent in the first step forms the nucleic acid based self-assembled complexes at a known concentration, the target nucleic acids that hybridize with the first nucleotides so that the nucleic acid based self-assembled complexes are not formed or are disassembled can be detected and quantified in the fourth step.

The intensity of the optical signal is strongest when the amount of the target nucleic acids that can be measured with the target nucleic acid detecting agent that forms the nucleic acid based self-assembled complexes at a known concentration is 0 or not greater than the minimum value, and the intensity of the optical signal decreases as the amount of the target nucleic acids increases. Therefore, whenever the optical signal of the nucleic acid-containing liquid sample is measured in the third step, the maximum intensity of the optical signal, which can be used as a reference can be secured, and the target nucleic acids can be quantitatively detected on the basis of a reduction change of the optical signal after the hybridization reaction in the second step.

The signal intensity is maximum when the amount of the target nucleic acid is less than the minimum detection sensitivity for the target nucleic acid of the target nucleic acid detecting agent of the first step, and the signal intensity is minimum when the amount of the target nucleic acid is greater than the maximum detection sensitivity for the target nucleic acid of the target nucleic acid detecting agent of the first step.

Accordingly, after the hybridization reaction of the second step, the target nucleic acid is quantitatively detected according to a change in intensity of the optical signal which decreases in a certain pattern depending on the amount of the target nucleic acid.

Accordingly, the analyzing algorithm of the fourth step is characterized by deriving detection and/or quantitative data of the target nucleic acid which interferes the formation of the nucleic acid based self-assembled complexes, which is calculated from detection and/or quantitative data determined from an optical signal derived from the nucleic acid based self-assembled complex being in Brownian motion in liquid. In addition, the analyzing algorithm of the fourth step may be obtained by machine learning.

In short, the third and fourth steps in the present invention are a turn-off method in which when the number of target nucleic acids that hybridize with the first nucleotide is 0 or less than the minimum detection sensitivity for the target nucleic acid of the agent of the first step, the intensity of the optical signal derived from the nucleic acid based self-assembled complex becomes the maximum value serving as a reference point, and the intensity of the optical signal derived from the nucleic acid based self-assembled complex being in Brownian motion in liquid decreases in a predetermined pattern according to the number of target nucleic acids that hybridize with the first nucleotide in the nucleic acid-containing liquid sample.

As shown in FIG. 5, in the case of the turn-off method, the signal intensity is maximum when the number of target nucleic acids is minimum. Therefore, the turn-off method can solve the problem with the turn-on method that can only perform qualitative detection (presence/absence) because there is no signal to be used as a reference each time the signal of the sample is measured.

As illustrated in FIG. 5, the signal intensity is maximum when the amount of target nucleic acid is minimum, and the signal intensity is minimum when the amount of target nucleic acid is maximum.

That is, the signal intensity is maximum when the amount of the target nucleic acid is less than the minimum detection sensitivity for the target nucleic acid of the agent of the first step, and the signal intensity is minimum when the amount of the target nucleic acid is greater than the maximum detection sensitivity for the target nucleic acid of the agent of the first step.

The detection sensitivity of the agent for the target nucleic acid in the first step is a reference point (Min, Max) of the light signal for each concentration of the nucleic acid based self-assembled complex in the target nucleic acid detecting agent, and the intensity of the optical signal becomes minimum when the amount of the target nucleic acids corresponding to a known concentration of the nucleic acid based self-assembled complexes is excessive.

In addition, the minimum and maximum reference points of an optical signal for each concentration of the nucleic acid based self-assembled complexes that can be formed in the target nucleic acid detecting agent may also be a subject for machine learning.

When the first metal nanoparticle and the second metal nanoparticle are metal nanoparticles exhibiting a localized surface plasmon resonance (LSPR) phenomenon, when the nucleic acid based self-assembled complex is designed such that a Raman indicator is disposed in a distance of 3 nm or less to the metal nanoparticle so as to amplify a Raman scattering signal through strong electromagnetic field enhancement by an action that the light emitted upon formation of the nucleic acid based self-assembled complexes causes a plasmon resonance phenomenon on the surface of the nanoparticle, a method for detecting a target nucleic acid according to one embodiment of the present invention measures the Raman shift value of the Raman indicator in Raman spectroscopy using a localized surface plasmon resonance (LSPR) phenomenon caused by metal nanoparticles through Raman spectroscopy for measuring the Raman shift value of a Raman indicator, and is a method in which when the number of target nucleic acids is 0 or less than the minimum detection sensitivity for the target nucleic acid of the target nucleic acid detecting agent in the first step, the Raman scattering signal derived from the nucleic acid based self-assembled complex being in Brownian motion in liquid has the maximum value serving as a reference point, and the intensity of the Raman scattering signal decreases with an increase in the amount of the target nucleic acid. The method can detect or quantify a target nucleic acid having a sequence complementary to that of a first nucleotide probe.

The target nucleic acid in a sample can be quantified through the minimum detectable concentration (threshold) of each target nucleic acid detecting agent.

A method for detecting a target nucleic acid according to one embodiment of the present invention can measure, by size change, the concentration of a nucleic acid based self-assembled complex that is self-assembled by complementary hydrogen bonding of a first nucleotide and a second nucleotide when the nucleic acid based self-assembled complex includes (a) a first nanoparticle based structure in which the first nucleotide which is a probe hybridizing with a target nucleic acid is linked to a first metal nanoparticle, and (b) a second nanoparticle based structure in which the second nucleotide which is complementary to the first nucleotide is linked to a second metal nanoparticle, through nanoparticle tracking analysis (NTA) that measures a particle size and a concentration by capturing light scattered from a particle being in Brownian motion, whereby the method can quantify the target nucleic acid which hybridizes with the first nucleotide so that the nucleic acid based self-assembled complex is not formed, or is disassembled.

In the method of detecting a target nucleic acid in liquid, according to the present invention, the presence or absence of an inhibitor causing a false negative result can be confirmed by adding a small known amount of a target nucleic acid to a sample.

When the target nucleic acid is a genome or a fragment thereof, the method of detecting a target nucleic acid in liquid according to the present invention can be used as a genome quantification and/or qualitative analysis method. That is, the method of detecting a target nucleic acid in liquid, according to the present invention, is an analysis method capable of identifying, qualitatively analyzing, and/or quantifying a genome or a pathogen using a hybridization method. In addition, it is possible to identify viruses and/or microorganisms or diagnose diseases through the detection of target nucleic acids.

[Target Nucleic Acid Detecting Agent]

The present invention provides a target nucleic acid detecting agent which detects a target nucleic acid by a turn-off method by using a reproducible optical signal derived from a nucleic acid based self-assembled complex being in Brownian motion in liquid.

The target nucleic acid detecting agent that detects a target nucleic acid by a turn-off method by using a reproducible optical signal derived from a nucleic acid based self-assembled complex being in Brownian motion in liquid, according to the present invention, features that
the nucleic acid based self-assembled complex includes: (a) a first nanoparticle based structure in which at least one first nucleotide, which is a probe being hybridized with the target nucleic acid depending on a condition, is linked to a first metal nanoparticle, and (b) a second nanoparticle based structure in which at least one second nucleotide which is 10 bp or more complementary to the first nucleotide is linked to a second metal nanoparticle, wherein the nucleic acid based self-assembled complex is self-assembled through complementary hydrogen bonds between the first nucleotide and the second nucleotide, wherein the nucleic acid based self-assembled complex is designed to detect a scattering signal that changes when the nucleic acid based self-assembled complex is not formed, or is disassembled, by hybridization of the first nucleotide and the target nucleic acid when the target nucleic acid exists.

Therefore, with the use of the target nucleic acid detecting agent of the present invention, it is possible to confirm whether the nucleic acid based self-assembled complex is formed and/or determine the degree of formation (quantification) of the nucleic acid based self-assembled complex by using a reproducible optical signal captured by the nucleic acid based self-assembled complex being in Brownian motion, and it is possible to detect or quantify the target nucleic acid that hybridizes with the first nucleotide so that the nucleic acid based self-assembled complex is not formed or is disassembled.

In addition, since the nucleic acid based self-assembled complex serves as a turn-off signal sensor when the target nucleic acid exists,
the target nucleic acid detecting agent that forms the nucleic acid based self-assembled complex at a known concentration is configured such that the intensity of the optical signal decreases with an increasing amount of the target nucleic acid while the intensity has a maximum value when the target nucleic acid does not exist, the intensity of the optical value has a minimum value when an excessive amount of the target nucleic acid corresponding to the known concentration of the nucleic acid based self-assembled complex exists, and
the minimum and maximum reference points of an optical signal for each concentration of the nucleic acid based self-assembled complex that can be formed in the target nucleic acid detecting agent can be secured and estimated.

In this case, the reference points (Min, Max) of the optical signal for each concentration of the nucleic acid based self-assembled complex that can be formed in the target nucleic acid detecting agent can also be a subject for machine learning.

[Characterization of Metal Nanoparticles Exhibiting Localized Surface Plasmon Resonance (LSPR)]

Metal nanoparticles are actively used in the field of in vivo and in vitro diagnostics due to their excellent durability and unique physical, chemical, and electrochemical properties depending on their size. The signal that is variable depending on the material, shape, and size of the metal nanoparticle has the advantage of being able to generate a stable signal for a long time because a unique signal can be transmitted without using an additional labeling material. Another advantage of metal nanoparticles is that they can amplify the signal of a fluorescent material, a small molecular weight indicator, etc. on the basis of the material properties of metals. For example, the plasmon resonance phenomenon of metal nanoparticles have an effect of amplifying optical properties such as Raman scattering signals and fluorescent molecular signals.

In addition, the surface-modified metal nanoparticles can improve sensor functions such as amplification of electrochemical signals and enhancement of sensitivity and selectivity. In addition, metal nanoparticles are used as sensors and are diversely used in clinics, drugs, and cancer treatment delivery systems.

Metal nanoparticles exhibiting localized surface plasmon resonance (LSPR) can be synthesized as metal nanoparticles, biofunctionalized metal nanoparticles, metal nanocomposites, or nanohybrids.

[Nucleic Acid Based Self-Assembled Complex]

In the present invention, the nucleic acid based self-assembled complex of turn-off based way for detecting a target nucleic acid is characterized by structural stability that produces reproducible scattering optical signal even while being in Brownian motion in liquid.

Accordingly, the nucleic acid based self-assembled complex formed in the target nucleic acid detecting agent of the present invention includes (a) a first nanoparticle based structure in which at least one first nucleotide, which is a probe being hybridized with a target nucleic acid depending on a condition, is linked to a first metal nanoparticle and (b) a second nanoparticle based structure in which at least one second nucleotide that is 10 bp or more complementary to the first nucleotide is linked to a second metal nanoparticle, and the nucleic acid based self-assembled complex is self-assembled by complementary hydrogen bonds of the first nucleotide and the second nucleotide. Here, diversely modified nucleic acid based self-assembled complexes fall within the scope of the present invention as long as they can serve as a sensor capable of detecting or quantifying a target nucleic acid in a turn-off way.

In order to design the nucleic acid based self-assembled complex of the present invention to maximize the signal/noise ratio depending on the presence or absence of the target nucleic acid, the metal nanoparticles are linked to the first nucleotide and/or the second nucleotide, and/or the first nucleotide or the second nucleotide is labeled with a Raman indicator, and/or magnetic nanoparticles may be linked to the second nucleotide.

As non-limiting examples of the nucleic acid based self-assembled complex of the present invention, a NEW structure, an ANEW structure, and a NEW Sight structure, which will be described later, are illustrated in FIGS. 1 and 2.

The NEW structure, ANEW structure, and NEW Sight structure are also nanostructures that enhance the S/N ratio according to the presence or absence of the target nucleic acids, and can increase resolution in terms of formation or non-formation/number/concentration of the nucleic acid based self-assembled complexes.

1. Nanoparticle Enhanced Wad (NEW) Structure

The nucleic acid based self-assembled complex of turn-off based way for detecting a target nucleic acid, according to one embodiment of the present invention is a nucleic acid based self-assembled complex (NEW structure) including: (a) a first nanoparticle based structure in which a first nucleotide which is a probe being hybridized with the target nucleic acid is linked to a first metal nanoparticle, and (b) a second nanoparticle based structure in which a second nucleotide that is complementary to the first nucleotide is linked to a second metal nanoparticle, wherein the nucleic acid based self-assembled complex is self-assembled through complementary hydrogen bonds between the first nucleotide and the second nucleotide,
wherein when the nucleic acid based self-assembled complex is formed, (i) a nanogap is formed by adjacent first and second metal nanoparticles, (ii) the nanogap is designed to be a space which generates and further enhances a surface plasmon resonance phenomenon when irradiated with light, and (iii) the nucleic acid based-assembled complex is designed to enhance a Raman scattering signal derived from the Raman indicator when irradiated with light, by disposing the Raman indicator attached to the first nucleotide or the second nucleotide in the nanogap.

In the NEW structure of the present invention, the nanogap is a crevice formed by the first metal nanoparticle and the second metal nanoparticle that are almost in almost contact. The Raman indicator may be disposed in the nanogap having a size of 3 nm or less, and/or the Raman indicator may be in contact with the first metal nanoparticle and/or the second metal nanoparticle.

In Raman spectroscopy, the NEW structure, which is a nucleic acid based self-assembled complex that acts as a turn-off signal sensor when a target nucleic acid exists, can directly quantify the target nucleic acid through measurement of a Raman scattering signal. In addition, it is easy to form a nanogap that maximizes the Raman scattering signal. When used as a detecting agent and/or a standard reagent, the structure is stable against temperate fluctuations in a range of 4° C. to 50° C. There is provided a measurement method in which when a target nucleic exists, the intensity is lower than that of the Raman scattering signal of the standard reagent which is a negative control (no target nucleic acid exists). Since a quantification interval can be set for each concentration of a nucleic acid based self-assembled complex that can be formed in the target nucleic acid detecting agent, a customized quantitative threshold can be applied during diagnosis. Therefore, the probability of false positive determination can be significantly reduced, and the trend of virus increase and decrease for the drug can be intuitively known.

According to one embodiment of the present invention, a method of detecting a target nucleic acid using a reproducible Raman scattering signal derived from the above-described nucleic acid based self-assembled complex (NEW structure) includes:
a first step of preparing a target nucleic acid detecting agent containing or forming the aforementioned nucleic acid based self-assembled complex (NEW structure);
a second step of heating to a temperature at which a complementary hydrogen bond between a first nucleotide and a second nucleotide is released,
and then cooling to a temperature at which the second nucleotide, if present, competes with the target nucleic acid and hybridizes with the first nucleotide probe; and
a third step of measuring a Raman scattering signal of a Raman indicator through Raman spectroscopy; and
a fourth step of providing detection and/or quantitative data of the target nucleic acid in a sample, through an algorithm that analyzes the Raman scattering signal measured in the third step.

1-1. Surface-Enhanced Raman Spectroscopy (SERS)

Since the aforementioned nucleic acid based self-assembled complex (NEW structure) is a nucleic acid based self-assembled complex designed to use surface-enhanced Raman spectroscopy (SERS), which is also called surface-enhanced Raman scattering, it corresponds to a kind of nucleic acid based self-assembled complex for Raman detection.

In order to most effectively enhance Raman emission, there must be a collective oscillation of free electrons on the metal surface between the metal and the incident light, which is called “surface plasmon”, which is the basis of an electromagnetic enhancement effect. That is, the incident light causes surface plasmon (electromagnetic effect) on the surface of the metal, and the surface plasmon interacts with the Raman indicator adsorbed on the metal surface (charge transfer effect), thereby significantly increasing the Raman emission (Phenomenon 1).

Furthermore, the surface-enhanced Raman scattering (SERS) phenomenon can be more strongly exhibited in the nanogap formed between metal nanoparticles capable of exhibiting localized surface plasmon resonance (LSPR) (Phenomenon 2).

Therefore, in order to solve the problem that it is difficult to measure a trace amount of substance by Raman spectroscopy because a Raman scattering signal has a weak intensity when measuring a trace amount of substance according a Raman scattering signal of a Raman indicator of which a Raman shift value is known, the aforementioned nucleic acid based self-assembled complex (NEW structure) is designed such that the two phenomena are used to generate and enhance a surface plasmon resonance phenomenon (electromagnetic effect), thereby amplifying a weak Raman scattering signal of the Raman indicator so that the intensity of the Raman scattering signal is increased to be 10⁴ to 10⁶ folds than a normal Raman signal intensity, and enhancing the S/N ratio depending on presence or absence of the target nucleic acid having an inverse functional relation with the formation of a nanogap to increase the resolution of quantification of the target nucleic acid.

In short, the nucleic acid based self-assembled complex (NEW structure) for Raman detection in the present invention is a nucleic acid based self-assembled complex designed to measure the Raman shift value of a Raman indicator by using an optical signal in Raman spectroscopy using localized surface plasmon resonance (LSPR) caused by metal nanoparticles.

The advanced nanoparticle enhanced wad (ANEW) structure to be described later is an improvement of the NEW structure for Raman to enhance the S/N ratio according to the presence or absence of a target nucleic acid, and the difference between these structures is illustrated in FIG. 2B.

According to one embodiment of the present invention, a target nucleic acid detecting agent containing or forming a NEW structure for Raman or an advanced nanoparticle enhanced wad (ANEW) structure which is an improvement of the NEW structure uses surface-enhanced Raman spectroscopy (SERS). When a Raman indicator is adsorbed on the surface of a nanogap formed by metal nanoparticle based structures or is disposed in a distance of several nanometers, due to the surface plasmon provided by the nanogap of the metal nanoparticle based structure, the Raman scattering of the Raman indicator, which has an intensity that is 10⁴-fold to 10⁶-fold increased compared to a normal Raman intensity that appears in the case where no nanogap exists is measured.

In this case, the number of nucleic acid based self-assembled complexes (NEW structures and ANEW structures) for Raman detection, that is, the number of nanogaps formed and the strength of the enhanced Raman scattering signal formed therefrom, have a function relationship with the number of target nucleic acids to be measured. Therefore, the target nucleic acid detecting agent forming the above-described nucleic acid based self-assembled complex can serve as an on/off signal system sensor, and can quantify the target nucleic acid through a computer algorithm on the basis of the intensity of the Raman scattering signal measured when irradiated with light.

The nucleic acid based self-assembled complex (NEW structure and ANEW structure) for Raman detection according to the present invention includes (a) a first nanoparticle based structure in which a first nucleotide which is a probe being hybridized with a target nucleic acid is linked to a first metal nanoparticle, and (b) a second nanoparticle based structure in which a second nucleotide that is 10 bp or more complementary to the first nucleotide is linked to a second metal nanoparticle, wherein the nucleic acid based self-assembled complex is self-assembled through a complementary hydrogen bond between the first nucleotide and the second nucleotide. In the nucleic acid based self-assembled complex (NEW structure and ANEW structure) for Raman detection according to the present invention, a nanogap is reproducibly formed between the first metal nanoparticle and the second metal nanoparticle that exhibit localized surface plasmon resonance (LSPR), a Raman indicator with known Raman shift value is disposed in the nanogap, and the Raman scattering signal of the Raman indicator can be further amplified by the nanogap. Even though being in in Brownian motion in liquid, it is possible to provide structural stability capable of reproducibly continuously providing Raman scattering signals that are amplified by the nanogap during measurement through 10 bp or more complementary hydrogen bonds of the first nucleotide and the second nucleotide.

In addition, another feature of the present is that when forming a nanogap of the first metal nanoparticle and the second metal nanoparticle which generate and further enhance the surface plasmon resonance phenomenon (electromagnetic effect), only metal nanoparticles that are preliminarily uniformly synthesized are used to reproducibly provide uniform nanogaps formed at regular intervals of several nanometers by the metal nanoparticles.

That is, in the newly designed nucleic acid based self-assembled complex for Raman detection according to the present invention, by the complementary hydrogen bond (i.e., spontaneous non-covalent bond) between the first nucleotide and the second nucleotide, the first metal nanoparticle and the second metal nanoparticle that are adjacent to each other form a nanogap that generates and further enhances the surface plasmon resonance phenomenon (electromagnetic effect). In this case, only spherical metal nanoparticles that are uniformly preliminarily synthesized to reproducibly provide uniform nanogaps having a crevice form having a size of several nanometers are used, thereby forming nanogaps each of which is precisely structurally defined between two metal nanoparticles.

On the other hand, as illustrated in FIGS. 1 and 2, the first nucleotide, which is a probe that hybridizes with a target nucleic acid in a target nucleic acid detecting agent containing or forming a nucleic acid based self-assembled complex for Raman detection according to the present invention, attaches to a target nucleic acid in a complementary manner when the target nucleic acid is present in an analysis subject. Thus, complementary hydrogen bonding between the first nucleotide and the second nucleotide cannot be made. For this reason, the nanogap between the first metal nanoparticle and the second metal nanoparticle which exhibit localized surface plasmon resonance (LSPR) cannot be formed or be assembled. Therefore, the amplification of the Raman scattering signal by the nanogap of the Raman indicator attached to either the first nucleotide or the second nucleotide is turned off.

Therefore, a target nucleic acid detecting agent containing or forming a nucleic acid based self-assembled complex for Raman detection at a known concentration can quantify a target nucleic acid through acquisition or estimation of reference points (Min, Max) (FIG. 5) for a Raman scattering signal intensity for each concentration of a nucleic acid based self-assembled complex therein, using the principle that the intensity of a Raman scattering is maximum when the target nucleic acid does not exist, the intensity of the Raman scattering signal decreases with an increasing amount of the target nucleic acid, and the intensity of the Raman scattering signal is minimum when an excessive amount of the target nucleic acid corresponding to a known concentration of the nucleic acid based self-assembled complex exists.

Alternatively, a nucleic acid based self-assembled complex for Raman detection, which is identical to the nucleic acid based self-assembled complex for Raman detection used as a detecting agent is used as a standard reagent, and a measurement method in which the intensity is reduced compared to the Raman scattering signal of the standard reagent is used to quantitatively detect a target nucleic acid (for example, a genome or a fragment thereof) having a complementary sequence to the first nucleotide probe.

In addition, the nucleic acid based self-assembled complex for Raman detection according to the present invention disposes a Raman indicator in a nanogap that exhibits a localized surface plasmon resonance (LSPR) phenomenon that amplifies the Raman scattering signal, thereby enhancing the Raman scattering signal. In order to ensure reproducibility, the nucleic acid based self-assembled complex serves as an on/off signal system sensor in which formation or non-formation of a nanogap is determined by an inverse functional relation with the presence or absence of the target nucleic acid to be measured. That is, the nucleic acid based self-assembled complex for Raman detection according to the present invention is a nanoparticle structure having high sensitivity and reproducibility for detection of a single DNA. This enables quantitative analysis of the target nucleic acid.

On the other hand, according to the present invention, in order to accurately dispose a Raman indicator in the nanogap between the first metal nanoparticle and the second metal nanoparticle that generate a local surface plasmon resonance (LSPR) to amplify the Raman scattering signal,
(1) the distance of the nanogaps formed by the metal nanoparticles is adjusted to be a specific range by adjusting the size and shape (preferably perfectly spherical) of the first metal nanoparticle and the second metal nanoparticle,
(2) in each of (a) a first nanoparticle-based structure in which a first nucleotide, which is a probe that hybridizes with a target nucleic acid, is linked to a first metal nanoparticle, and (b) a second nucleotide complementary to the first nucleotide is linked to a second metal nanoparticle, the length of a spacer that is linked to the oligonucleotide attacher attached to the metal nanoparticles and which helps complementary hydrogen bonding of 10 bp or more by increasing the degree of freely moving to help complementary is adjusted, the binding position of the Raman indicator on the spacer in the first nucleotide or the second nucleotide may be adjusted.

Therefore, the present invention can quantify and/or qualitatively analyze a target nucleic acid material that has an inverse functional relation with whether or not the nucleic acid based self-assembled complex for Raman detection of the present invention is (re)formed, that is, whether or not to (re)form a nanogap, with high selectivity and sensitivity even though the amount of the target nucleic acid is trace, from an amplified intensity of a Raman scattering signal from an Raman shift value of a Raman indicator corresponding to the wavelength of an incident laser beam used for Raman analysis, by using (i) the structural characteristics of the nanogap formed between metal nanoparticles and (ii) a localized surface plasmon resonance (LSPR) phenomenon that is generated or enhanced by the relationship between incident light rays during Raman analysis, in which (i) and (ii) are implemented in the nucleic acid based self-assembled complex for Raman detection of the present invention. Due to this, multiple detection of target nucleic acids is possible using amplified Raman scattering signal intensities at Raman shift values of various Raman indicators corresponding to the wavelength of laser incident light used in Raman analysis.

In addition, the nucleic acid based self-assembled complex for Raman detection of the present invention can be used not only as a target nucleic acid detecting agent, but also as a negative control in which a target nucleic acid to be measured does not exist when Raman spectroscopy is performed. In addition, it can be used as a standard reagent for Raman analysis in which the concentration of the nucleic acid based self-assembled complex for Raman detection of the present invention is known. On the other hand, since the concentration of the nucleic acid based self-assembled complex for Raman detection of the present invention is known before a sample containing a target nucleic acid to be detected is added, Raman spectroscopy can be performed before the sample containing the target nucleic acid to be detected is added, and it is used as a standard reagent for this.

1-2. Operation Principle of Nucleic Acid Based Self-Assembled Complex for Raman Detection

The nucleic acid based self-assembled complex for Raman detection of the present invention is designed such that the first nucleotide probe has a complementary sequence to the target nucleic acid to be detected, and the second nucleotide is designed to be identical to a part of the sequence of the target nucleic acid. Therefore, after heating a liquid sample containing the nucleic acid based self-assembled complex for Raman detection of the present invention to a temperature at which the complementary hydrogen bond between the probe and the second nucleotide is released, the second nucleotide competes with the target nucleic acid again to carry out the hybridization reaction with the first nucleotide probe. Therefore, on the basis of the number (i.e., number of nanogaps) of the nucleic acid based self-assembled complexes for Raman detection, which are reformed through complementary hydrogen bonding of the first nucleotide probe with the second nucleotide rather than the second nucleotide, it is possible to determine whether the target nucleic acid exists in a liquid sample or to determine the concentration thereof (FIGS. 5 and 6).

That is, when the target nucleic acid hybridizes with the first nucleotide probe through complementary hydrogen bonds, the opportunity for the second nucleotide to form complementary hydrogen bonds with the first oligonucleotide probe is lost. Therefore, the nucleic acid based self-assembled complex for Raman detection of the present invention is not re-formed, so that the nanogap disappears, and the Raman scattering signal of the Raman indicator is not amplified (FIG. 1). The number of the nucleic acid based self-assembled complexes (corresponding to the number of nanogaps) of the present invention, which are re-formed through complementary hydrogen bonds between the first nucleotide probes and the second nucleotides decreases by the number that is equal to the number of target nucleic acids existing in a measurement sample. Therefore, the number of target nucleic acids contained in the measurement sample is in proportional to a decreased amount of the intensity of a Raman scattering signal of a Raman indicator compared to a standard reagent in Raman spectroscopy. In this case, (i) an agent of which the concentration of the nucleic acid based self-assembled complexes for Raman detection of the present invention is known is used as a standard reagent for Raman spectroscopy and for calculation of the number of the re-formed nucleic acid based self-assembled complexes of the present invention, and (ii) as a negative control that does not contain the target nucleic acids to be measured, before or after a sample containing the target nucleic acids to be detected is added, a sample that contains the nucleic acid based self-assembled complexes for Raman detection and which is not yet heated to a temperature at which the complementary hydrogen bond between the first nucleotide probe and the second nucleotide is released can be used as a standard reagent for Raman spectroscopy measurement and for calculation of the number of re-formed (re-assembled) nucleic acid based self-assembled complexes for Raman detection. In this case, the nucleic acid based self-assembled complex for Raman detection of the present invention can serve as a standard reagent for calculation of the number of nucleic acid based self-assembled complexes for Raman detection and a target nucleic acid detecting agent.

1-3. Raman Indicator

In the present invention, the Raman indicator is a substance that serves as a marker that can determine whether the first nucleotide and the second nucleotide hybridize. For example, it may be Cy3, Cy5, Rhodamine 6G, 44DP (4,4′-Bipyridine) whose Raman shift value is known.

The Raman indicator is a substance that facilitates detection and measurement of an analyte by a Raman detection device, and the Raman indicator may be a compound whose Raman shift value is known.

In the nucleic acid based self-assembled complex (NEW structure) of the present invention, the nanogap is a crevice formed by the contact between the first metal nanoparticle and the second metal nanoparticle. To make the Raman indicator able to be disposed in the nanogap having a size of 3 nm or less, and/or the Raman indicator able to be almost in contact with the first metal nanoparticle and/or the second metal nanoparticle, the Raman indicator may attach to an oligo nucleotide spacer directly or via any linker compound (for example, C1-2 compound).

For example, the second nucleotide is a nucleotide in which a Raman indicator of which a Raman shift value is known, is linked between (i) the 2-1 oligonucleotide attacher having a nucleic acid sequence complementary to that of the oligonucleotide probe of the first nucleotide and (ii) the spacer; or the first nucleotide is a nucleotide in which a Raman indicator of which a Raman shift value is known, is linked between (i) the oligonucleotide probe having a nucleic acid sequence that hybridizes with a part of the sequence of the target nucleic acid and (ii) the spacer.

Raman indicators may be organic or inorganic molecules, atoms, complexes or synthetic molecules, dyes, naturally occurring dyes (such as phycoerythrin), organic nanostructures such as C60, buckyballs, carbon nanotubes, quantum dots, organic fluorescent molecules, and the like. Specifically, non-limiting examples of the Raman indicator include FAM, Dabcyl, tetramethyl rhodamine-5-isothiocyanate (TRITC), malachite green isothiocyanate (MGITC), X-Rhodamine-5-isothiocyanate (XRITC), 3,3-diethylthiadicarbocyanine iodide (DTDC), tetramethyl rhodamine isothiol (TRIT), 7-nitrobenz-2-1,3-diazole (NBD), phthalic acid, terephthalic acid, isophthalic acid, para-aminobenzoic acid, erythrosine, biotin, digoxigenin, 5-carboxy-4′,5′-dichloro-2′,7′-dimethoxy, fluorescein, 5-carboxy-2′,4′,5′,7′-tetrachlorofluorescein, 5-carboxyfluorescein, 5-carboxy rhodamine, 6-carboxyrhodamine, 6-carboxytetramethyl amino phthalocyanine, azomethine, cyanine (Cy3, Cy3.5, Cy5), xanthine, succinyl fluorescein, aminoacridine, quantum dot, carbon allotrope, cyanide, thiol, chlorine, bromine, methyl, phosphorus, and sulfur. The Raman indicator needs to exhibit a clear Raman spectrum, and is preferably organic fluorescent molecules including cyanine-based fluorescent molecules such as Cy3, Cy3.5, Cy5, or FAM, Dabcyl, and Rhodamine-based fluorescent molecules. Organic fluorescent molecules have the advantage of being able to detect higher Raman scattering signals by resonating with an excitation laser wavelength used in Raman analysis.

1-4. Metal Nanoparticles

In the nucleic acid based self-assembled complex (NEW structure) for Raman detection, the first metal nanoparticle and the second metal nanoparticle are preferably particles causing a surface plasmon resonance (LSPR) phenomenon that amplifies the scattering signal of the Raman indicator disposed in the nanogap formed between the first metal nanoparticle and the second metal nanoparticle.

The two fundamental mechanisms for enhancement of Raman scattering signals are electromagnetic enhancement and chemical enhancement. In particular, for significant enhancement, electromagnetic effects play a leading role. This electromagnetic enhancement depends on the roughness characteristics of the metal surface, such as the presence of nanogaps formed by the metal nanoparticle based structures. For this reason, surface-enhanced Raman scattering signals mainly appear on analytes adsorbed on the surface of coinage metals such as gold, silver, copper, etc., or alkali metals such as lithium, sodium, and potassium, which have an excitation wavelength in the visible or near visible ray region.

The intensity of the Raman scattering signal is proportional to the square of the electromagnetic field applied to the analyte, and the electromagnetic field is expressed as the sum of the electromagnetic field applied to the analyte in the absence of roughness and the electromagnetic field generated from the roughness of a particulate metal. Accordingly, highly surface-enhanced Raman scattering signals can be provided by controlling the surface structure, that is, the size and shape of the metal nanoparticles.

That is, the metal nanoparticles can amplify the production of Raman scattering signals of a Raman indicator such as a fluorescent material, a small molecular weight indicator, etc. on the basis of the material properties of metals. For example, the plasmon resonance phenomenon of metal nanoparticles has an effect of amplifying optical properties such as Raman scattering signals and fluorescent molecular signals.

In general, the physical and chemical properties of metal nanoparticles, including optical and electrical properties, can be controlled through changes in size, shape, crystal structure, and the like. In particular, noble metal nanoparticles made of gold, silver, etc., strongly resonate with light in the visible wavelength region, resulting in very strong light absorption and scattering.

The frequency of surface plasmon resonance varies depending on the type of metal nanoparticle (usually Au, Ag, Cu, Pt, Pd, etc.), size and shape, dispersion solvent, and type of laser (incident light). Therefore, a more sensitive surface-enhanced Raman scattering signal can be obtained by controlling such factors.

In the present invention, the metal nanoparticles may each independently be AuNP, AuNNP, CRD, Au@Ag, or Ag@Au. A gold or silver shell may be formed on magnetic nanoparticles.

The first metal nanoparticle and the second metal nanoparticle may be the same or different in size, shape, material, and structure.

With the use of the nucleic acid based self-assembled complex having a nanogap of several nanometers, a Raman scattering signal can be significantly enhanced to increase the S/N ratio. However, when the spacing between nanoparticles is increased, an effect of enhancing a Raman scattering signal cannot be obtained. Therefore, the nanogap used in surface-enhanced Raman spectroscopy is formed to have a size of 10 nm or less, and preferably 3 nm or less. In this case, the size of the nanogap may be controlled by adjusting the sizes of the metal nanoparticles.

The metal nanoparticles, i.e., the first metal nanoparticle and the second metal nanoparticle, each independently preferably have a diameter of 5 nm to 300 nm. When the diameter is smaller than 5 nm, there is a problem in that the Raman surface enhancement effect is not significant, whereas when the diameter exceeds 300 nm, biological applications are limited. More preferably, the diameter is preferably in a range of from 10 nm to 40 nm. The metal nanoparticles may be approximately spherical in shape, but nanoparticles of any shape (for example, irregular shape) can be used.

When the size of the first metal nanoparticles is different from the size of the second metal nanoparticles, larger nanoparticles have an average diameter of 30 to 50 nm, and smaller nanoparticles have an average diameter of 20 to 40 nm.

Preferably, the metal nanoparticles may be nanoparticles made of metal, metal oxide, or metal nitride. In addition, the material of the metal nanoparticles is preferably selected from the group consisting of Au, Ag, Cu, Pt and Pd, and alloys thereof to enhance the Raman scattering signal of a Raman indicator connected thereto by providing an electromagnetic field enhanced by surface plasmon resonance.

In the present invention, the metal nanoparticle is a metal nanoparticle to the surface of which the first nucleotide and the second nucleotide are directly attached, and is preferably made of gold or silver.

In the case of gold nanoparticles, surface modification is easy through the Au-thiol reaction, and a large amount of material can be attached to the surface of the nanoparticles.

Gold nanoparticles can form stable bonds with various organic molecules on their surface, and maintain stable bonds even at high physiological salt concentrations at which biomaterials (such as nucleotides and proteins) can maintain their original structures. Accordingly, gold nanoparticles modified with the first nucleotide can form a strong hydrogen bond with a target nucleic acid having a nucleotide sequence complementary to the first nucleotide, enabling detection of the target nucleic acid. That is, various target nucleic acid sequences can be detected with a very low detection limit by using the characteristics of gold nanoparticles and the complementary hydrogen bonding characteristics of nucleotides. However, gold nanoparticles have a disadvantage that the Raman signal amplification (SERS) effect is smaller than that of silver nanoparticles.

On the other hand, silver nanoparticles have the advantage of having excellent Raman scattering effect, but have the disadvantage of low stability at high salt concentration and high temperature at which biological materials can maintain their original structure. Gold-silver alloy nanoparticles have the disadvantage of low stability because irreversible aggregation occurs at or above a high salt concentration (0.3 M NaCl) at which oligonucleotides are hybridized.

In the case of a gold/silver core-shell structure, since silver nanoparticles form a shell, there is an advantage that the Raman amplification effect can be used. It has been reported that a silver/gold core-shell structure can hardly detect a Raman signal but a gold/silver core-shell structure can sensitively detect a Raman signal.

For example, in the case of using metal nanoparticles having a gold-core/silver-shell structure,
a first nucleotide in which (i) an oligonucleotide probe having a nucleic acid sequence that hybridizes with a target nucleic acid, (ii) a spacer, and (iii) an oligonucleotide attacher attaching to a first metal nanoparticle are sequentially covalently linked; or
a second nucleotide in which (i) a 2-1 oligonucleotide attacher having a nucleic acid sequence complementary to that of the oligonucleotide probe of the first nucleotide, (ii) a spacer, and (iii) a 2-2 oligonucleotide attacher attaching to a second nanoparticle are sequentially covalently linked,
is attached to a metal core by the oligonucleotide attacher attaching to the first metal nanoparticle, or the 2-2 oligonucleotide attacher, and then a silver shell is formed.

Meanwhile, it is important to synthesize high-purity nanomaterials that are uniform in physical and chemical characteristics, surface charge, and shape thereof. In the case of using nanoparticles, size control is easy, various materials can be used depending on the purpose, and a large amount of material can be synthesized in a relatively easy way because in many cases they are synthesized in an aqueous solution.

Gold nanoparticles may be used with or without the addition of a surface stabilizer. In addition, the gold nanoparticles may be used in a state of being dispersed in an organic solvent or aqueous solution. Preferably, the gold nanoparticles are used in a state of being dispersed in an aqueous solution. Gold nanoparticles have strong affinity with various organic molecules and can form stable bonds with organic molecules. Therefore, the first nucleotide and/or the second oligonucleotide can be bound to the surface of the gold nanoparticles via an oligonucleotide attacher.

Meanwhile, the surface of the metal nanoparticles may be modified with a protective oligonucleotide so that the metal nanoparticles may not be aggregated and may thus be well dispersed.

Of the details of the NEW structure described above, redundant parts will be omitted when describing the ANEW structure and the NEW Sight structure.

2. Advanced Nanoparticle Enhanced was (ANEW) Structure

A nucleic acid based self-assembled complex of turn-off based way for detecting a target nucleic acid according to one embodiment of the present invention,
includes (a) a first nanoparticle based structure in which a first nucleotide, which is a probe hybridizing with a target nucleic acid is linked to a first metal nanoparticle, and (b) a second nanoparticle based structure in which a second nucleotide that is complementary to the first nucleotide is linked to a second metal nanoparticle that is magnetic, in which the nucleic acid based self-assembled complex is self-assembled through complementary hydrogen bonding between the first nucleotide and the second nucleotide,
when the nucleic acid based self-assembled complex is formed, (i) a nanogap is formed by the first and second metal nanoparticles adjacent to each other, (ii) the nanogap is designed to serve as a space which generates and further enhances a surface plasmon resonance phenomenon when irradiated with light, and (iii) the nucleic acid based self-assembled complex is designed to enhance a Raman scattering signal derived from a Raman indicator when irradiated with light, by disposing the Raman indicator attached to the first nucleotide or the second nucleotide in the nanogap.

The ANEW structure is a nanostructure with an increased S/N ratio compared to the NEW structure, and thus has improved quantification performance.

For example, the second metal nanoparticles that are magnetic may be gold-coated magnetic particles.

The second nanoparticle based structure in which the second nucleotide is linked to the second metal nanoparticle that is magnetic is collected or recovered using a magnet, regardless that it is self-assembled with the first nanoparticle based structure in which the first nucleotide is linked to the first metal nanoparticle. The filtrate remaining after the removal of all the magnetic second metal nanoparticles contains the first nanoparticle based structure in which the first nucleotide is linked to the first metal nanoparticle, in which only the first nanoparticle based structure hybridized with the target nucleic acid rather than the second nucleotide is contained. Therefore, only the Raman scattering signal of the Raman indicator linked to the first metal nanoparticle can be measured.

In the case of the method of detecting a target nucleic acid in a liquid phase using the above-described NEW structure, the complementary hydrogen bond between the first nucleotide and the second nucleotide is broken (denatured) (b) so that the second nanoparticle based structure in which the second nucleotide is linked to the second metal nanoparticle coexists in the Raman measurement space. However, in the case of the method of detecting a target nucleic acid in a liquid phase using the ANEW nanostructure, since the second nanoparticle based structure in which the second nucleotide competing with the target nucleic acid is linked to the second metal nanoparticle that is magnetic is clearly removed by a simple method, the effect of increasing the S/N ratio is expected in Raman measurement.

4. Nanoparticle Enhanced Wad Sight Algorithm (NEW Sight) Structure

A nucleic acid based self-assembled complex of turn-off based way for detecting a target nucleic acid according to one embodiment of the present invention (NEW Sight structure),
is a nucleic acid based self-assembled complex including: (a) a first nanoparticle based structure in which a first nucleotide, which is a probe hybridizing with a target nucleic acid is linked to a first metal nanoparticle, and (b) a second nanoparticle based structure in which a second nucleotide that is complementary to the first nucleotide is linked to a second metal nanoparticle, in which the nucleic acid based self-assembled complex is formed by complementary hydrogen bonding between the first nucleotide and the second nucleotide.

The concentration of the nucleic acid based self-assembled complex can be measured through nanoparticle tracking analysis (NTA) that measures and visualizes particle sizes and concentrations by capturing light scattered from particles being in Brownian motion.

The NEW Sight structure can provide number-based nanomaterial concentration and high-resolution size distributions through nanoparticle tracking analysis (NTA).

Since the NEW Sight structure measures the concentration for each of the metal nanoparticles through scattering light derived from the first metal nanoparticles and/or the second metal nanoparticles, the Raman indicator is not required. In addition, a modification in which the first metal nanoparticles and the second metal nanoparticles are made of a non-metal material also falls within the scope of the present invention, as long as the non-metal material is a nanoparticle.

The NEW Sight structure can provide particle-by-particle optical signals regardless of whether an indicator is used or not through scattering light derived from the nucleic acid based self-assembled complex being in Brownian motion. Accordingly, by capturing light scattered from particles being in Brownian motion, the nucleic acid based self-assembled complexes provide high-resolution particle-by-particle size data and concentration measurements for colloidal suspensions or nanoparticle solutions.

An algorithm that analyzes the optical signal or a change value thereof measured and provides detection and/or quantitative data of the target nucleic acid contained in the sample may be an algorithm that makes a determination by tracking changes in size.

Unlike the NEW structure, it tracks changes in size of the structure by performing measurement without using a Raman scattering signal but using a particle analyzer such as NanoSight. Therefore, a Raman indicator is not required, and a measurement method is very simple. Since it does not use fluorescent or Raman scattering signals, it does not require Raman indicators (for example, Cy3, Cy5, etc.).

The structure of a dimer that is self-assembled through complementary hydrogen bonding between the first nucleotide and the second nucleotide is simpler than that of the NEW structure described above. When using NanoSight, target genome detection and quantitative results can be obtained simply.

[Light Source and Raman Detection Device]

Laser light is single-wavelength in-phase light. In general, laser beams are thin and do not spread. Lasers are mainly used in the field of spectroscopy because of their precisely defined monochromatic wavelengths.

Fundamentally, the disadvantage of Raman spectroscopy is that the intensity of the signal is weak. Therefore, it is preferable to use a laser capable of providing high-power incident light, that is, high-density photons, as a light source. Therefore, it is preferable to have a photomultiplier tube (PMT), an avalanche photodiode (APD), a charge coupled device (CCD), or the like that can effectively amplify a detection signal, as a detector.

In the present invention, (i) the Raman surface enhancement effect by metal nanoparticles using localized surface plasmon resonance (LSPR), (ii) Raman scattering signal intensity amplification level of a Raman indicator further amplified by nanogaps and/or (iii) the Raman shift value of the Raman indicator may vary depending on the wavelength of a laser beam used in Raman analysis.

In the method of detecting a target nucleic acid of the present invention, the third step of obtaining a Raman scattering signal of a Raman indicator through Raman spectroscopy may be performed by any known Raman spectroscopy method. Preferably, surface-enhanced Raman spectroscopy (SERS), surface enhanced resonance Raman spectroscopy (SERRS), hyper-Raman and/or coherent anti-Stokes Raman spectroscopy (CARS) may be used.

Compared to the fluorescence signal, the Raman scattering signal has a fingerprinting signal pattern for each molecule. Therefore, it is possible to clearly detect each of nano-sized materials. A molecular detection method using such a Raman scattering signal is a precise and stable non-destructive molecular measurement method widely used in the field of physical chemistry and the like. Since light (laser) is used, when a sufficient signal is generated, one measurement time is relatively short, for example, about 1 to 10 seconds. Since molecules are directly measured using light without a gene amplification process, expensive materials such as fluorescent markers, polymerases, and primers are not required for gene amplification, so it is relatively inexpensive. Furthermore, even unskilled users can use it immediately after familiarizing themselves with simple usage.

Any suitable form or configuration of Raman spectroscopy or related techniques known in the art can be used for analyte detection. Examples thereof include, but are not limited to, normal Raman scattering, resonance Raman scattering, surface-enhanced Raman scattering, surface-enhanced resonance Raman scattering, incoherent anti-Stokes Raman spectroscopy (CARS), stimulated Raman scattering, inverse Raman spectroscopy, stimulated gain Raman spectroscopy, hyper-Raman scattering, molecular optical laser examiner (MOLE), or Raman microprobe, or Raman microscopy, or confocal Raman microspectroscopy, three-dimensional or scanning Raman, Raman saturation spectroscopy, time resolved resonance Raman, Raman dissociation spectroscopy, or UV-Raman microscopy.

In the present invention, the Raman detection device may include a computer. The embodiments do not impose any restrictions on the type of computer used. An exemplary computer may include an information communication bus and a processor for processing information. The computer may further include RAM or another type of dynamic storage device, ROM or another type of static storage device, and a data storage device such as magnetic or optical disks and corresponding drives. Additionally, the computer may include peripheral devices known in the art, such as a display device (for example, a cathode ray tube or a liquid crystal display), an alphabetic input device (for example, a keyboard), a cursor control device (for example, a mouse, a trackball, or cursor direction keys), and a communication device (for example, modems, network interface cards, or interface devices used to couple with Ethernet, Token Ring, or other types of networks).

In the present invention, the Raman detection device may be operably coupled with the computer. Data transmitted from the detection device may be processed by the processor and the data may be stored in a main memory. Data on release profiles for standard analytes may also be stored in the main memory or ROM. The processor can identify the analyte type of a sample by comparing the emission spectrum exhibited from the analyte on a Raman active substrate. The processor may analyze the data transmitted from the detection device to determine the identity and/or concentration of various analytes. Differently equipped computers may be used for specific implementations. Accordingly, the system structure may vary from embodiment to embodiment of the present invention. After data collection, the data will typically be transmitted for a subsequent data analysis task. To facilitate the analysis task, data obtained by the detection device will be analyzed by a typical method using a digital computer as described above. Typically, the computer will be suitably programmed for receiving and storing data from the detection device, as well as analyzing and reporting the collected data.

### Advantageous Effects

The present invention can detect a target nucleic acid in a turn-off based way by using a reproducible optical signal derived from a nucleic acid based self-assembled complex which is in Brownian motion in liquid. Therefore, quantification and/or qualitative analysis of a genome can be performed using a hybridization method.

### BRIEF DESCRIPTION OF DRAWINGS

FIG. 1 is a diagram illustrating the principle of operation in which a nucleic acid based self-assembled complex (NEW structure) for Raman detection according to one embodiment of the present invention serves as a turn-off signal system sensor in the presence of a target nucleic acid.

FIG. 2 is a diagram schematically showing the design and formation principles of various nucleic acid based self-assembled complexes according to the present invention: (A) NEW structures, (B) ANEW structures, and (C) NEW Sight structures.

FIG. 3 is a graph of the results of 100 repeated measurements of Raman signals generated from the NEW structure prepared in Example 1.

FIG. 4 is a still image of a video taken by a nanoparticle tracking analyzer (NTA) to show that two adjacent metal nanoparticles in the NEW structure prepared in Example 1 perform Brownian motion.

FIG. 5 shows a Raman signal generated from the NEW structure prepared in Example 1, in which the left graph shows a negative control (NC) state in which the NEW structure is present intact without dissociation in the absence of a target nucleic acid, and the right graph shows a positive control (PC) state in which the structure is completely dissociated in the presence of an excessive amount of target nucleic acids.

FIG. 6 shows a Raman signal obtained by using the NEW structure prepared in Example 1, in which the signal decreases with an increasing amount of the target nucleic acids by a turn-off method.

FIG. 7 is a schematic diagram showing the principle of Raman scattering.

FIG. 8 is a schematic diagram of propagating plasmon and localized surface plasmon resonance (LSPR).

FIG. 9 is an exemplary view of Raman spectrum information of a sample: (a) one or more Raman shift values, (b) the highest Raman point at each shift, and (c) the lowest Raman point at each shift.

FIG. 10 is a schematic diagram illustrating the operation principle of a biosensor using metal nanoparticles exhibiting localized surface plasmon resonance (LSPR).

### BEST MODE

Hereinafter, the present invention will be described in detail. However, the following examples are only intended to clearly illustrate the technical features of the present invention, but do not limit the protection scope of the present invention.

### Example 1: [Preparation of Target Nucleic Acid Detecting Agent Containing Nucleic Acid Based Self-Assembled Complex]

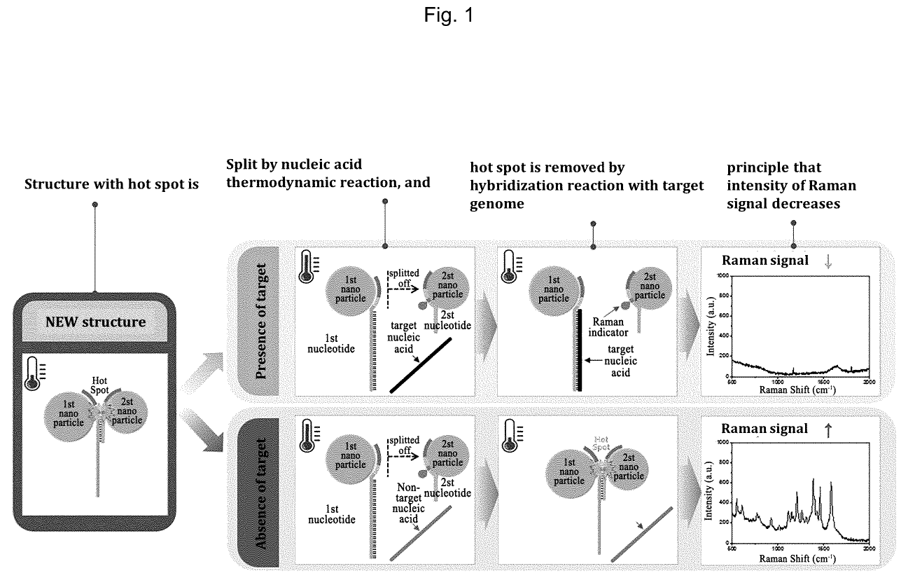

Referring to FIG. 1, a target nucleic acid detecting agent prepared in Example 1 contains nucleic acid based self-assembled complexes (NEW structures). The NEW structure includes (a) a first nanoparticle based structure in which a first nucleotide to hybridize with a target nucleic acid is linked to a spherical gold metal nanoparticle having a diameter of 20 to 30 nm, and (b) a second nanoparticle based structure in which a second nucleotide that is complementary to the first nucleotide is linked to a spherical gold nanoparticle having a diameter of 20 to 30 nm. The nucleic acid based self-assembled complex is formed by self-assembly through complementary hydrogen bonding between the first nucleotide and the second nucleotide in a water-based solvent.

In this case, the target nucleic acid was synthesized to have a nucleotide sequence (12 mer to 30 mer) that can be identified as the genome of a corona virus that is currently pandemic.

An oligonucleotide probe having a nucleotide sequence that hybridizes with the target nucleic acid, a C3 spacer represented by Formula 1 below that helps complementary hydrogen bonding of 10 bp or more by increasing the degree of freely moving, and an oligonucleotide attacher (poly-adenine 10mer) that attaches to a nanoparticles 10mer were sequentially linked to prepare the first nucleotide to hybridize with the target nucleic acid.

In addition, a second nucleotide complementary to the first nucleotide was prepared by sequentially linking a 2-1 oligonucleotide attacher having a base sequence that is 20 to 50 bp complementary to that of the oligonucleotide probe of the first nucleotide, a C3 spacer represented by Formula 2 below and helping 10 bp or more complementary hydrogen bonding by increasing the degree of freely moving, and a 2-2 oligonucleotide attacher (poly-adenine 10mer) attaching to a nanoparticle. Here, Cy3 as a Raman indicator was disposed between the C3 spacer in the second nucleotide and the 2-1 oligonucleotide attacher.

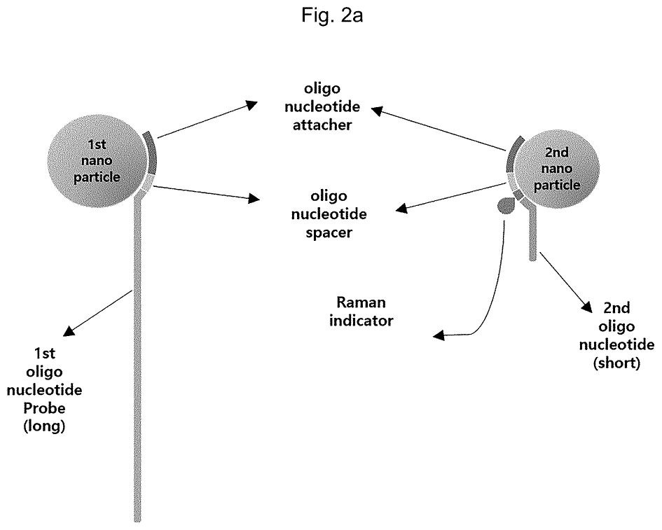

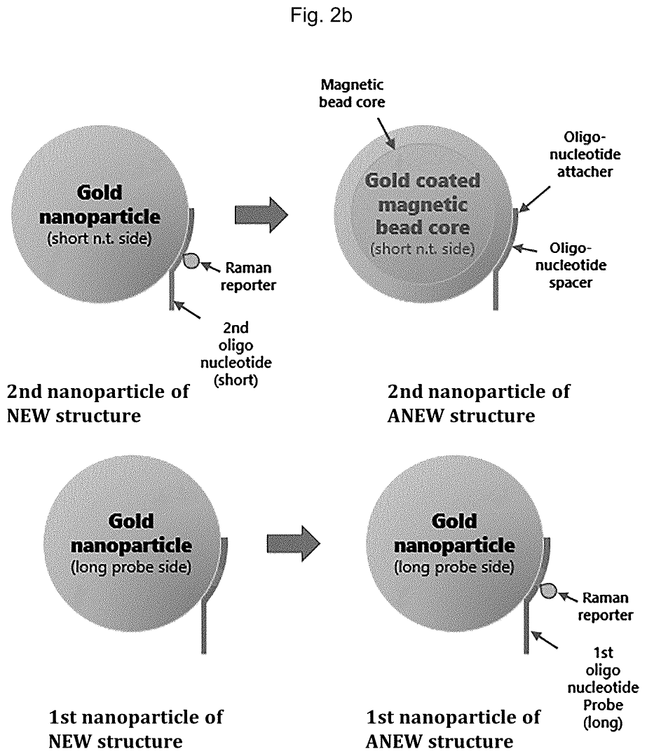

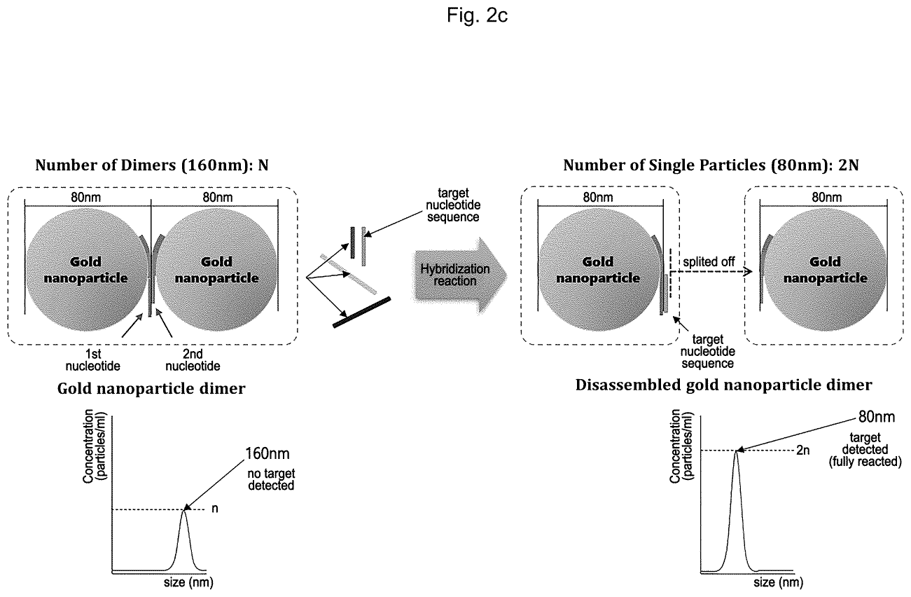

However, the sequence length of the 2-1 oligonucleotide attacher is shorter than the sequence length of the synthesized target nucleic acid so that the target nucleic acid gains an advantage in competition when hybridizing with the first nucleotide (FIGS. 1 and 2).

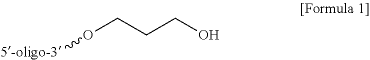

The first nanoparticle based structure was synthesized by sequentially adding 100 mM phosphate buffer and 2M NaCl to a mixed solution of gold nanoparticles and the first nucleotide modified with a —SH group at one end and allowing reaction at room temperature. Likewise, the second nanoparticle based structure was synthesized by sequentially adding 100 mM phosphate buffer and 2M NaCl to a mixed solution of gold nanoparticles and the second nucleotide modified with a —SH group at one end and allowing reaction at room temperature.

Subsequently, an aqueous solution containing the first nanoparticle based structures and an aqueous solution containing the second nanoparticle based structures were mixed, to induce complementary hydrogen bonding between the first nucleotide and the second nucleotide to form nucleic acid based self-assembled complexes. Thus, a target nucleic acid detecting agent containing the nucleic acid based self-assembled complexes was prepared.

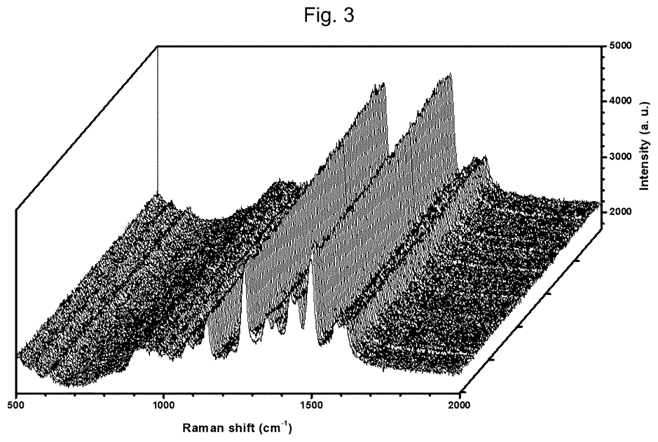

Using a custom-made inverted Raman detection device, the Raman signal of the NEW structure, that is, the Raman signal of the Raman indicator Cy3 linked to the second nucleotide was measured in the target nucleic acid detecting agent containing the NEW structures in the phosphate buffer prepared in Example 1. The results are shown in FIG. 3.

Scattering Raman spectra were recorded in the range of 500 to 2000 cm-1 at 400 μW, one acquisition, and one second tracking (accumulation). Despite the low intensity, the characteristic peaks of Cy3 appeared at 1470 cm-1 and 1580 cm-1, which are fingerprint spectra at 532 nm laser incident light.

Surprisingly, it was found that the above-described NEW structure in the target nucleic acid detecting reagent reproducibly provided a stably enhanced Raman scattering signal within a certain range even when continuously measured 100 times per second.

### Example 2: Concentration Determination of Nucleic Acid Based Self-Assembled Complexes Using a Nanoparticle Tracking Analyzer (NTA)

A nanoparticle tracking analyzer (NTA) available from Malvern Panalytical was used to determine the concentration of the NEW structures in the NEW structure-containing target nucleic acid detecting agent prepared in Example 1.

The nanoparticle tracking analyzer (NTA) used in the present example has functions of (i) tracking each particle, by measuring the Brownian motion of each particle individually and simultaneously and causing particle diffusion over time, (ii) measuring particle sizes, by converting the particle diffusion into magnitudes using the Stokes-Einstein equation and providing a high-solution data set without size bias, (iii) and providing reproducible concentration-related high-precision data through a smart algorithm. In addition, since the sizing principle in the nanoparticle tracking analyzer (NTA) is absolute, no calibration is required. Each particle is independently sized and measured simultaneously, enabling in-depth understanding of even the most complex samples. The device accurately detects the smallest changes in particle size, thereby rapidly providing information about events such as aggregation within a population. This accuracy and sensitivity are very important given the physicochemical properties of materials that are intrinsically linked to their size at the nanoscale.

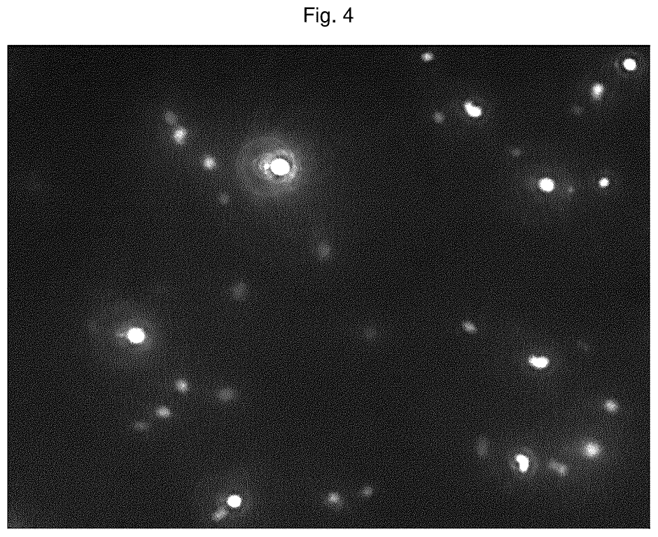

Through a nanoparticle tracking analyzer (NTA) that measures and visualizes particle sizes and concentrations by capturing light scattered from particles being in Brownian motion, the concentration of the above-described nucleic acid based self-assembled structures (NEW structures) in the target nucleic acid detecting agent of the example can be measured. In addition, as illustrated in FIG. 4, it was observed through a video that the two adjacent metal nanoparticles in the target nucleic acid detecting agent containing the above-described nucleic acid based self-assembled complexes (NEW structures) prepared in Example 1 perform Brownian motion (FIG. 4).

### Example 3: Measurement of Raman Signal after Hybridization with a Target Nucleic Acid

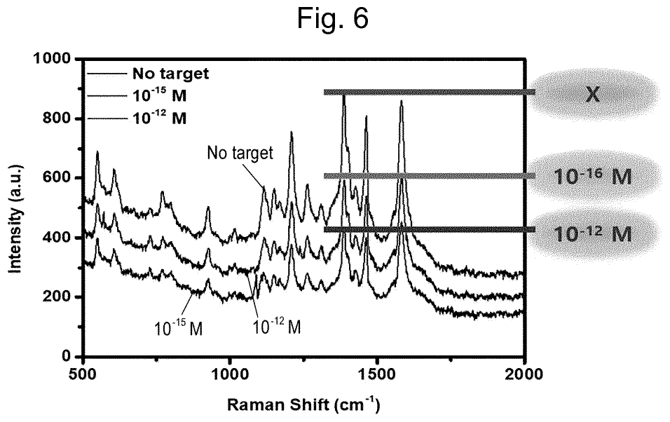

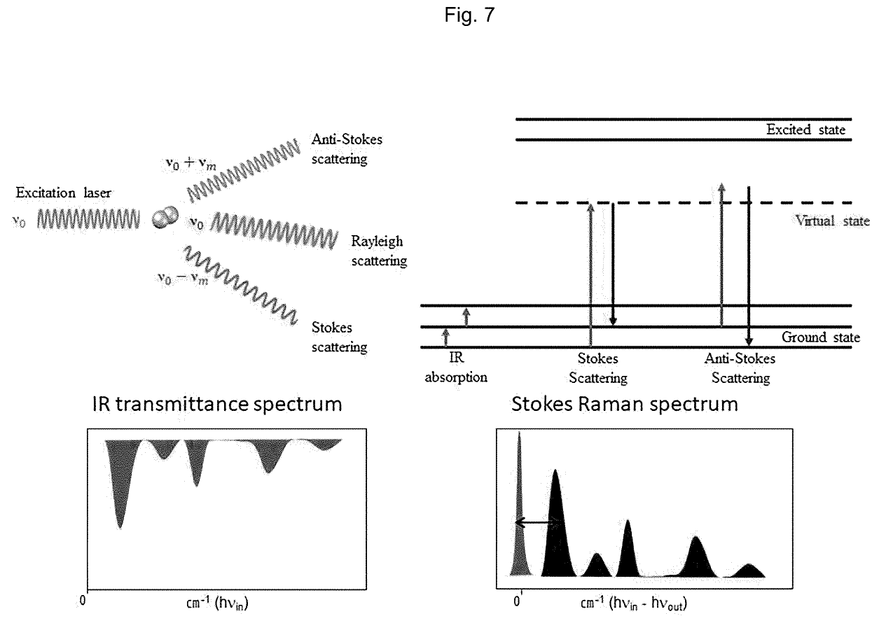

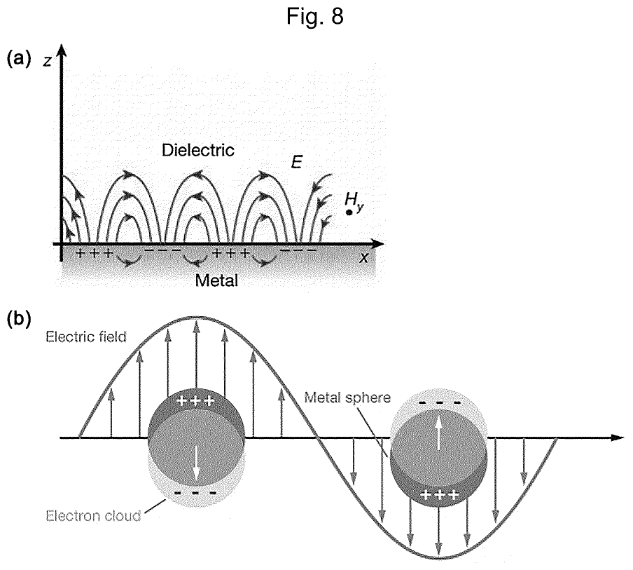

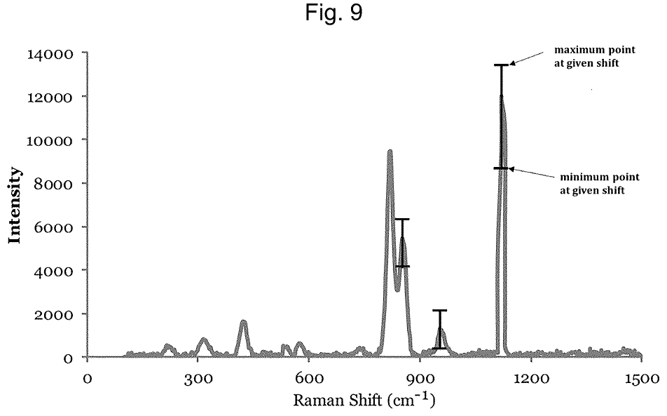

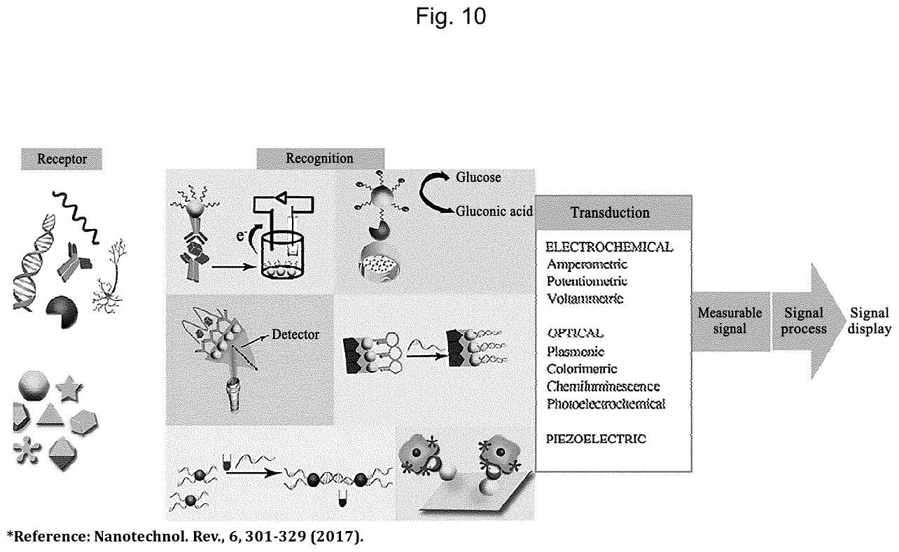

The nucleic acid based self-assembled complex (NEW structure) prepared in Example 1 is linked to a Raman indicator that exhibits an already-known Raman shift value for the second nucleotide competing with the target nucleic acid for hybridization with the first nucleotide, and the nucleic acid based self-assembled complex (NEW structure) serves as a turn-off signal system sensor in the presence of a target nucleic acid. Therefore, formation or non-formation/number/concentration of the nucleic acid based self-assembled complexes can be confirmed (quantitated) using a Raman signal derived from the Raman indicator (FIGS. 1 and 6).

The synthesized target nucleic acid (12 mer to 30 mer) was added at a known concentration to the target nucleic acid detecting agent containing the nucleic acid based self-assembled complexes (NEW structures) prepared in Example 1 (FIG. 6).

The target nucleic acid detecting agent was heated to a temperature Tm of 72.0° C. at which the nucleic acid based self-assembled complex formed by complementary hydrogen bonding of a first nucleotide and a second nucleotide respectively provided in (a) a first nanoparticle based structure in which the first nucleotide hybridizing with a target nucleic acid is linked to a spherical gold nanoparticle and (b) a second nanoparticle based structure in which the second nucleotide complementary to the first nucleotide is linked to a spherical gold nanoparticle is disassembled, i.e., temperature at which the complementary hydrogen bond between the first nucleotide and the second nucleotide is denatured. Then, the temperature was lowered by 5° C. That is, the target nucleic acid detecting agent was cooled to a hybridization temperature Ta at which the first nucleotide and the target nucleic acid hybridize with each other.

An inverted Raman detection device self-manufactured in the same manner as in Example 1 was used to measure the Raman signal of the Raman indicator linked to the second nucleotide.

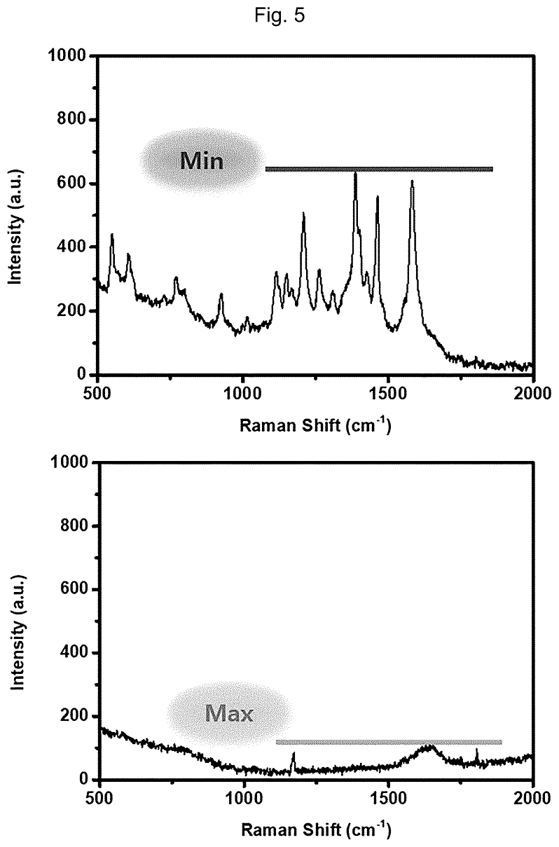

The left graph in FIG. 5 is a Raman spectrum measured after raising the temperature of the target nucleic acid detecting agent that contains no target nucleic acid to 72.0° C. and then lowering the temperature by about 5° C., and the right graph in FIG. 5 is a Raman spectrum measured when a target nucleic acid was added in an excess amount compared to the number of the first nucleotides contained in the target nucleic acid detecting agent.

The number of nucleic acid based self-assembled complexes formed by spontaneous molecular-level hydrogen bonding between the first nucleotide, which is a probe that hybridizes with the target nucleic acid, and the second nucleotide complementary to the first nucleotide, has an inverse functional relation with the number of target nucleic acids (FIG. 1). That is, (i) when the target nucleic acids in the sample are absent or present in an amount that is below the lower limit of the detection sensitivity of the target nucleic acid detecting agent for the target nucleic acids (the minimum value of the detection range of the target nucleic acid detecting agent containing or forming nucleic acid based self-assembled complexes), the number of nucleic acid based self-assembled complexes (NEW structures) formed by self-assembly of the first and second nucleotides that are complementary to each other has a maximum value, and (ii) when the target nucleic acids are present in an amount that is higher than the upper limit of the detection sensitivity of the target nucleic acid detecting agent for the target nucleic acids, the number of nucleic acid based self-assembled complexes (NEW structures) formed by self-assembly of the first and second nucleotides that are complementary to each other has a minimum value (FIG. 5).

The nucleic acid based self-assembled complexes (NEW structures) in the target nucleic acid detecting agent prepared in Example 1 serve as turn-off signal system sensors in the presence of target nucleic acids. That is, the target nucleic acid detecting agent containing an already-known concentration of nucleic acid based self-assembled complexes exhibits a Raman signal of the highest intensity when no target nucleic acids exist, exhibits a Raman signal of decreasing intensity with an increasing amount of the target nucleic acids, and exhibits a Raman signal of the lowest intensity in the presence of an excessive amount of target nucleic acids corresponding to the already-known concentration of nucleic acid based self-assembled complexes. Thus, it is possible to secure and estimate reference points (Min, Max) of an optical signal for each concentration of nucleic acid based self-assembled complexes in the target nucleic acid detecting agent.

FIG. 6 is a Raman spectrum measured when the synthesized target nucleic acid (12 mer to 30 mer) was added at known concentrations (0 M, 10-16 M, and 10-12 M) to the target nucleic acid detecting agent containing the nucleic acid based self-assembled complexes (NEW structures) prepared in Example 1 (FIG. 6).

Surprisingly, in the target nucleic acid detecting agent forming the above-described nucleic acid based self-assembled complexes (NEW structures), in the absence of the target nucleic acid or in the presence of the already-known concentration of the target nucleic acid, it was found that a Raman scattering signal upon light irradiation decreased in a predetermined pattern while having an inverse functional relation with the target nucleic acid concentration (FIG. 6). From the fact, it can be inferred that since the number of nucleic acid based self-assembled complexes (NEW structures) (i.e., the intensity of the enhanced Raman scattering signal resulting therefrom) has a functional relation with the number of target nucleic acids to be measured, the target nucleic acid detecting agent forming the above-described nucleic acid based self-assembled complex can serve as an on/off signal system sensor, and can quantify the target nucleic acid through a computer algorithm on the basis of the intensity of the Raman scattering signal measured when irradiated with light.

In conclusion, the target nucleic acid detecting agent as in Example 1 can measure a change (decrease) in Raman signal of a Raman indicator when a nucleic acid based self-assembled complex is not formed by hybridization of a first nucleotide and a target nucleic acid or is disassembled, and quantify the target nucleic acid (FIGS. 1 to 6).

1.-33. (canceled)

34. A gold nanoparticle complex comprising:
a first nanoparticle structure comprising a first gold nanoparticle, and a first oligonucleotide,
wherein a 3′-end of the first oligonucleotide is linked to the first gold nanoparticle; and
a second nanoparticle structure comprising a second gold nanoparticle, a Raman indicator, and a second oligonucleotide,
wherein a 5′-end of the second oligonucleotide is linked to the second gold nanoparticle via the Raman indicator,
wherein a length of a sequence of the second oligonucleotide is shorter than a length of a sequence of the first oligonucleotide,
wherein the second oligonucleotide has a sequence which is at least 10 nt complementary to a sequence of the first oligonucleotide;
wherein the Raman indicator is interposed between a gap between the first gold nanoparticle and the second gold nanoparticle,
wherein the gap is narrow enough that the localized surface plasmon resonance is occurred when the Raman indicator emits the Raman scattering signal.

35. A gold nanoparticle complex comprising:
a first nanoparticle structure comprising a first gold nanoparticle, and a first oligonucleotide,
wherein a 5′-end of the first oligonucleotide is linked to the first gold nanoparticle; and
a second nanoparticle structure comprising a second gold nanoparticle, a Raman indicator, and a second oligonucleotide,
wherein a 3′-end of the second oligonucleotide is linked to the second gold nanoparticle via the Raman indicator,
wherein a length of a sequence of the second oligonucleotide is shorter than a length of a sequence of the first oligonucleotide,
wherein the second oligonucleotide has a sequence which is at least 10 nt complementary to a sequence of the first oligonucleotide;
wherein the Raman indicator is interposed between a gap between the first gold nanoparticle and the second gold nanoparticle,
wherein the gap is narrow enough that the localized surface plasmon resonance is occurred when the Raman indicator emits the Raman scattering signal.

36. A gold nanoparticle complex for detecting a target nucleic acid, comprising:
a first nanoparticle structure comprising a first gold nanoparticle, and a first oligonucleotide,
wherein a 3′-end of the first oligonucleotide is linked to the first gold nanoparticle,
wherein the first oligonucleotide has a complementary sequence to a sequence of the target nucleic acid; and
a second nanoparticle structure comprising a second gold nanoparticle, a Raman indicator, and a second oligonucleotide,
wherein a 5′-end of the second oligonucleotide is linked to the second gold nanoparticle via the Raman indicator,
wherein the second oligonucleotide has a sequence which is at least 10 nt complementary to a sequence of the first oligonucleotide,
wherein a length of the second oligonucleotide is shorter than a length of the first oligonucleotide,
wherein a length of the complementary part of the second oligonucleotide to the first oligonucleotide is shorter than a length of the target nucleic acid;
wherein the Raman indicator is interposed between a gap between the first gold nanoparticle and the second gold nanoparticle,
wherein the gap is narrow enough that the localized surface plasmon resonance is occurred when the Raman indicator emits the Raman scattering signal.

37. A gold nanoparticle complex for detecting a target nucleic acid, comprising:
a first nanoparticle structure comprising a first gold nanoparticle, and a first oligonucleotide,
wherein a 5′-end of the first oligonucleotide is linked to the first gold nanoparticle,
wherein the first oligonucleotide has a complementary sequence to a sequence of the target nucleic acid; and
a second nanoparticle structure comprising a second gold nanoparticle, a Raman indicator, and a second oligonucleotide,
wherein a 3′-end of the second oligonucleotide is linked to the second gold nanoparticle via the Raman indicator,
wherein the second oligonucleotide has a sequence which is at least 10 nt complementary to a sequence of the first oligonucleotide,
wherein a length of the second oligonucleotide is shorter than a length of the first oligonucleotide,
wherein a length of the complementary part of the second oligonucleotide to the first oligonucleotide is shorter than a length of the target nucleic acid;
wherein the Raman indicator is interposed between a gap between the first gold nanoparticle and the second gold nanoparticle,
wherein the gap is narrow enough that the localized surface plasmon resonance is occurred when the Raman indicator emits the Raman scattering signal.

38. The gold nanoparticle complex of claim 34, wherein the gap between the first gold nanoparticle and the second gold nanoparticle is less than 10 nm.

39. The gold nanoparticle complex of claim 35, wherein the gap between the first gold nanoparticle and the second gold nanoparticle is less than 10 nm.

40. The gold nanoparticle complex of claim 36, wherein the gap between the first gold nanoparticle and the second gold nanoparticle is less than 10 nm.

41. The gold nanoparticle complex of claim 37, wherein the gap between the first gold nanoparticle and the second gold nanoparticle is less than 10 nm.
